(19) United States
(12) Patent Application Publication
(10) Pub. No.: US 2016/0081947 A1
(43) Pub. Date: Mar. 24, 2016

(54) SELECTIVE LSD1 AND DUAL LSD1/MAO-B INHIBITORS FOR MODULATING DISEASES ASSOCIATED WITH ALTERATIONS IN PROTEIN CONFORMATION

(71) Applicant: ORYZON GENOMICS S.A., Barcelona (ES)

(72) Inventors: Tamara MAES, Castelldefeis (ES); Carlos BUESA ARJOL, Castelldefeis (ES)

(21) Appl. No.: 14/675,990

(22) Filed: Apr. 1, 2015

Related U.S. Application Data
(63) Continuation of application No. 13/876,485, filed on Jul. 26, 2013, now abandoned.
(60) Provisional application No. 61/404,332, filed on Sep. 30, 2010.

(51) Int. Cl. A61K 31/135; A61K 31/495; A61K 31/165

(52) U.S. Cl. CPC A61K 31/135; A61K 31/165; A61K 31/495

(57) ABSTRACT

The invention relates to methods and compositions for the treatment or prevention of protein conformation disorders. In particular, the invention relates to an LSD1 inhibitor for use in treating or preventing a protein conformation disorder, such as, e.g., Huntington Disease.

### FIELD OF THE INVENTION

The invention relates to methods and compositions for the treatment or prevention of protein conformation disorders. In particular, the invention relates to an LSD1 inhibitor for use in treating or preventing a protein conformation disorder, such as, e.g., Huntington Disease.

### BACKGROUND OF THE INVENTION

Abnormal protein conformation such as aberrant protein folding and protein aggregates are hallmarks of many diseases including degenerative diseases. Although there are common denominators amongst many of these diseases, their outward manifestations appear to be dictated by tissue specific protein conformation defects. These diseases are typically late-onset, characterized by slow progressive deterioration, loss of nerve cells, and eventually leading to death.

One specific class of protein folding/aggregation disorders are referred to as CAG repeat disorders which includes at least ten distinct diseases. This class of disorder is associated with an expansion of the CAG nucleotide repeat which codes for glutamine (Q in single letter code) in specific genes (sometimes called trinucleotide repeat disorders). The specific gene that harbors the repeat dictates the type of disease. For example, Huntington disease is characterized by CAG expansion in the Huntington gene. Kennedy disease is characterized by a CAG expansion in the androgen receptor gene.

A number of spinocerebellar ataxias are also associated with CAG expansions and have CAG expansions in different ataxin genes. Typically, there is an inverse correlation between the age of onset in these diseases and the number of CAG repeats. Furthermore, the number of repeats seems to correlate with over severity of these diseases although not necessarily with each specific symptom of the disease. For example, Burk et al., report that cognitive dysfunction is not associated with repeat length in spinocerebellar ataxia 2 patients (Burk et al., Brain (1999) 122(4):769-777). Similar studies have been performed in spinocerebellar ataxia type 6 where again it was found that cognitive dysfunction is not correlated to repeat length. Thus, in some cases the motor effects and cognitive effects in these diseases are dissociable.

Another example of dissociation of symptoms with repeat length is found in HD where recently it was found that repeat length correlated with impairment on a number of scales including cognitive and motor scales but not behavioral (Ravina et al., Mov. Disord. (2008) Jul. 15; 23(9): 1223-7).

Aberrant gene expression in affected tissue as compared to normal tissue is a common characteristic of many human diseases. This is true for cancer and many neurological diseases which are characterized by changes in gene expression patterns. Gene expression patterns are controlled at multiple levels in the cell. Control of gene expression can occur through modifications of DNA: DNA promoter methylation is associated with suppression of gene expression. Several inhibitors of DNA methylation are approved for clinical use including the blockbuster Vidazam™.

Another class of modifications involve histones that form the protein scaffold that DNA is normally associated with (coiled around) in eukaryotic cells. Histones play a crucial role in organizing DNA and the regulated coiling and uncoiling of DNA around the histones is critical in controlling gene expression—coiled DNA is typically not accessible for gene transcription. A number of histone modification have been discovered including histone acetylation, histone lysine methylation, histone arginine methylation, histone ubiquinylation, and histone sumoylation, many of which modify accessibility to the associated DNA by the cells transcriptional machinery. These histone marks serve to recruit various protein complexes involved in transcription and repression. An increasing number of studies are painting an intricate picture of how various combinations of histone marks control gene expression in cell-type specific manner and a new term has been coined to capture this concept: the histone code.

The prototypical histone mark is histone acetylation. Histone acetyl transferase and histone deacetylases are the catalytic machines involved in modulation of this histone mark although typically these enzymes are parts of multiprotein complexes containing other proteins involved in reading and modifying histone marks. The components of these protein complexes are typically cell type and typically comprise transcriptional regulators, repressors, co-repressors, receptors associated with gene expression modulation (e.g., estrogen or androgen receptor). Histone deacetylase inhibitors alter the histone acetylation profile of chromatin. Accordingly, histone deacetylase inhibitors like SAHA, TSA, and many others have been shown to alter gene expression in various in vitro and in vivo animal models. Clinically, histone deacetylase inhibitors have demonstrated activity in the cancer setting and are being investigated for oncology indications as well as for neurological conditions and other diseases.

A group of enzymes known as histone lysine methyl transferases and histone lysine demethylases are involved histone lysine modifications. One particular human histone lysine demethylase enzyme called Lysine Specific Demethylase-1 (LSD1) was recently discovered (Shi et al. (2004), Cell 119:941) to be involved in this crucial histone modification. LSD1 has a fair degree of structural similarity, and amino acid identity/homology to polyamine oxidases and monoamine oxidases, all of which (i.e., MAO-A, MAO-B and LSD1) are flavin dependent amine oxidases that catalyze the oxidation of nitrogen-hydrogen bonds and/or nitrogen carbon bonds. Although the main target of LSD1 appears to be mono- and di-methylated histone lysines, specifically H3K4 and H3K9, there is evidence in the literature that LSD1 can demethylate methylated lysines on non-histone proteins like p53, E2F1, and Dnmt1.

Several groups have reported LSD1 inhibitors in the literature. Sharma et al. recently reported a new series of urea and thiourea analogs based on an earlier series of polyamines which were shown to inhibit LSD1 and modulate histone methylation and gene expression in cells (J. Med. Chem. (2010) PMID:20568780 [PubMed—as supplied by publisher]). Sharma et al. note that “To date, only a few existing compounds have been shown to inhibit LSD1.” Some efforts were made to make analogs of the histone peptide that is methylated by the enzyme, other efforts have focused on more small molecule-like molecules based on known MAO inhibitors. Gooden et al. reported trans-2-arylcyclopropylamine analogues that inhibit LSD1 with Ki values is the range of 188-566 micromolar (Gooden et al. ((2008) Bioorg. Med. Chem. Let. 18:3047-3051)). Most of these compounds were more potent against MAO-A as compared to MAO-B. Ueda et al. ((2009) J. Am. Chem. Soc. 131(48):17536-17537) reported cyclopropylamine analogs selective for LSD1 over MAO-A and MAO-B that were designed based on reported X-ray crystal structures of these enzymes with a phenylcyclopropylamine-FAD adduct and a FAD-N-propargyl lysine peptide. The reported IC50 values for phenylcyclopropylamine were about 32 micromolar for LSD1 whereas as compounds 1 and 2 had values of 2.5 and 1.9 micromolar, respectively.

Importantly, studies have also been conducted on amine oxidase inhibitor compounds to determine selectivity for MAO-A versus MAO-B since MAO-A inhibitors can cause dangerous side-effects (see, e.g., Yoshida et al. (2004), Bioorg. Med Chem. 12(10):2645-2652; Hruschka et al. (2008), Bioorg. Med Chem. (16):7148-7166; Folks et al. (1983), J. Clin. Psychopharmacol. (3)249; and Youdim et al. (1983), Mod. Probl. Pharmacopsychiatry (19):63).

Currently, the treatments available for these types of diseases yield only marginal benefits and are not thought to alter the course of the disease. There is a need for new drugs for these diseases that target novel points of intervention in the disease processes and avoid side-effects associated with certain targets. Furthermore, there is a need for compounds that have pharmacokinetic and toxicity profiles that are suitable from chronic treatment of protein conformation diseases, particularly neurodegenerative disorders.

### BRIEF SUMMARY OF THE INVENTION

The present invention relates to the treatment or prevention of protein conformation diseases or diseases associated with alterations in protein conformation. The inventors have unexpectedly found that selective inhibitors of LSD1 and dual inhibitors of LSD1 and MAO-B can ameliorate some symptoms of protein conformation disorders when administered chronically in amounts sufficient to inhibit LSD1 or LSD1/MAO-B. Advantageously, the use of selective LSD1 inhibitors or dual LSD1/MAO-B inhibitors avoids side-effects associated with targets such as MAO-A. The inventors found that administration of LSD1 inhibitors chronically was well tolerated in a mammal (and dual LSD1/MAO-B inhibitors). Thus, the inventors have unexpectedly found that selective LSD1 inhibition or LSD1/MAO-B dual inhibition is a new therapeutic approach to protein conformation diseases that is tolerable in mammals and alleviates or reduces the decline of certain symptoms of these diseases.

In one aspect, the invention is a method of treating or preventing a cognitive symptom in an individual having a protein conformation disorder comprising identifying a patient in need of such treatment and administering to said individual for a sufficient period of time an amount of an LSD1 inhibitor sufficient to improve the cognitive symptom or reduce the rate of decline of the cognitive symptom thereby treating or preventing said cognitive symptom.

In a related aspect, the invention is the use of an LSD1 inhibitor in an amount sufficient to modulate LSD1 activity for treating or preventing cognitive decline in a protein conformation disorder. In a specific aspect, cognitive decline in a protein conformation disorder refers to cognitive decline in a CAG expansion disease. In a more specific aspect, the CAG expansion disorder is Huntington Disease. In one embodiment of this aspect, the amount of selective LSD1 inhibitor administered is sufficient to modulate or inhibit LSD1 activity while not substantially inhibiting MAO-A activity, thereby avoiding or reducing side-effects associated with administration of MAO-A inhibitors.

In another aspect, the invention is a method of treating or preventing a motor symptom in an individual having a protein conformation disorder comprising identifying an individual in need of such treatment and administering to said individual for a sufficient period of time an amount of an LS1 inhibitor sufficient to reduce the rate of decline in said motor symptom thereby treating said motor symptom. In a related aspect, the invention is the use of an LSD1 inhibitor in an amount sufficient to modulate LSD1 activity for treating or preventing a motor symptom in a protein conformation disorder. In a specific aspect, cognitive decline in a protein conformation disorders refers to cognitive decline in a CAG expansion disease. In a more specific aspect, the CAG expansion disorder is Huntington Disease. In one embodiment of this aspect, the amount of selective LSD1 inhibitor administered is sufficient to modulate or inhibit LSD1 activity while not substantially inhibiting MAO-A activity, thereby avoiding or reducing side-effects associated with administration of MAO-A inhibitors.

In another aspect, the invention is a method of increasing longevity in an individual having a protein conformation disorder comprising identifying an individual in need of such treatment and administering to said individual for a sufficient period of time an amount of an LSD1 sufficient to increase longevity. In a related aspect, the invention is the use of an LSD1 inhibitor in an amount sufficient to modulate LSD1 activity for treating or preventing decreased longevity associated with a protein conformation disorder. In a specific aspect, cognitive decline in a protein conformation disorders refers to cognitive decline in a CAG expansion disease. In a more specific aspect, the CAG expansion disorder is Huntington Disease. In one embodiment of this aspect, the amount of selective LSD1 inhibitor administered is sufficient to modulate or inhibit LSD1 activity while not substantially inhibiting MAO-A activity, thereby avoiding or reducing side-effects associated with administration of MAO-A inhibitors.

In one aspect, the protein conformation disorder is a CAG expansion disorder.

In again another aspect, the protein conformation disorder is Alzheimer Disease.

In still another aspect, the protein conformation disorder is Parkinson Disease.

In one aspect, the CAG repeat disorder is Huntington disease, Kennedy Disease, Spinocerebellar Ataxia 1, Spinocerebellar Ataxia 2, Spinocerebellar Ataxia 3, Spinocerebellar Ataxia 6, Spinocerebellar Ataxia 7, or Spinocerebellar Ataxia 17.

In one aspect, the CAG repeat disorder is Huntington disease.

In one aspect, the sufficient period of time for administering the LSD1 inhibitors is from five or more days to the individual, more preferably from five days to four years, even more preferably from five days to two years, yet even more preferably for fifteen days to two years, and again yet even more preferably from fifteen days to one year. In one aspect, the LSD1 inhibitor is administered daily in amount sufficient to yield a Cmax above the IC50 value for the LSD1 inhibitor.

The invention also relates to an LSD1 inhibitor for use in any of the above-described methods.

Accordingly, the invention relates to an LSD1 inhibitor (or a pharmaceutical composition comprising an LSD1 inhibitor and a pharmaceutically acceptable carrier) for use in treating or preventing a protein conformation disorder. The invention also relates to an LSD1 inhibitor (or a pharmaceutical composition comprising an LSD1 inhibitor and a pharmaceutically acceptable carrier) for use in treating or preventing a cognitive symptom or cognitive decline in an individual (preferably a mammal; more preferably a human) having a protein conformation disorder. Likewise, the invention encompasses a an LSD1 inhibitor (or a pharmaceutical composition comprising an LSD1 inhibitor and a pharmaceutically acceptable carrier) for use in improving a cognitive symptom in an individual (preferably a mammal; more preferably a human) having a protein conformation disorder or for use in reducing the rate of decline of a cognitive symptom in an individual (preferably a mammal; more preferably a human) having a protein conformation disorder. The invention also relates to an LSD1 inhibitor (or a pharmaceutical composition comprising an LSD1 inhibitor and a pharmaceutically acceptable carrier) for use in treating or preventing a motor symptom in an individual (preferably a mammal; more preferably a human) having a protein conformation disorder. Moreover, the invention encompasses an LSD1 inhibitor (or a pharmaceutical composition comprising an LSD1 inhibitor and a pharmaceutically acceptable carrier) for use in increasing longevity in an individual (preferably a mammal; more preferably a human) having a protein conformation disorder or for use in treating or preventing decreased longevity associated with a protein conformation disorder.

The protein conformation disorder may, e.g., be a CAG expansion disease (or CAG expansion disorder or CAG repeat disorder), such as Huntington Disease, Kennedy Disease, Spinocerebellar Ataxia 1, Spinocerebellar Ataxia 2, Spinocerebellar Ataxia 3, Spinocerebellar Ataxia 6, Spinocerebellar Ataxia 7, or Spinocerebellar Ataxia 17. The protein conformation disorder may also be Alzheimer Disease or Parkinson Disease. The present invention particularly relates to the treatment or prevention of Huntington Disease using an LSD1 inhibitor.

The LSD1 inhibitor to be used in accordance with the invention is preferably a selective LSD1 inhibitor or a dual LSD1/MAO-B inhibitor.

### BRIEF DESCRIPTION OF THE DRAWINGS

FIG. 1: Optimization of Selective LSD1 Inhibitors. FIG. 1 summarizes structure-activity relationship evolution of increased potency towards LSD1 as compared to MAO-A and/or MAO-B from compounds that were not selective (e.g., tranylcypromine) to compounds that are selective inhibitors of LSD1 with IC50 values in the low nanomolar range.

FIG. 2: Optimization of Dual LSD1/MAO-B Inhibitors. FIG. 2 summarizes structure-activity relationship evolution of increased potency towards LSD1 and MAO-B as compared to MAO-A from compounds that were not selective for LSD1 and MAO-B (e.g., tranylcypromine). The dual LSD1/MAO-B compounds have IC50 values for these two targets in the low nanomolar range.

FIG. 3: Compound Dual-1 Increases Histone Methylation. FIG. 3A shows the results of a western blot stained for H3K4 methylation with SH-SY5Y cells grown in the presence of Compound Dual-1 (100 μM) or parnate (“PNT”) (250 μM) for one, two, and three days, showing that this compound, Dual-1, increases levels of dimethylated H3K4 in cells in a time dependent manner. FIG. 3B is a graph showing quantification of the results shown in FIG. 3A.

FIG. 4: Dual LSD1/MAO-B inhibitors Attenuate Eye Degeneration in Huntington Flies. FIG. 4 shows results of studies in fly lines expression a mutant Huntington gene in the eye of Drosophila. Treatment with Compound Dual-1, a dual inhibitor of LSD11/MAO-B, reduced rhabdomere degeneration as compared to vehicle treated flies (FIG. 4A) at day 7. Each triplet of bars in the graph represents from left to right (1) vehicle treated cells, (2) cells treated with 1 μM of Compound Dual-1, and (3) cells treated with 5 μM of Compound Dual-1. Y-axis is frequency (%) and X-axis is the number of remaining rhabdomeres. Treatment with Compound Dual 2, a dual inhibitor of LSD1/MAO-B, reduced rhabdomere degeneration as compared to vehicle treated flies (FIG. 4B). Each triplet of bars in the graph represents from left to right (1) vehicle treated cells, (2) cells treated with 1 μM of Compound Dual-2, and (3) cells treated with 5 μM Dual-2. Y-axis is frequency (%) and X-axis is the number of remaining rhabdomeres.

FIG. 5: Compound Selective-1 (selective LSD1 inhibitor) Attenuates Eye Degeneration in Fly. FIG. 5 shows results of studies in fly lines expression a mutant Huntington gene in the eye of Drosphila. Treatment with Compound Selective-1, a selective inhibitor of LSD1, reduced rhadomere degeneration as compared to vehicle treated flies at day 2. Each triplet of bars in the graph represents from left to right (1) vehicle treated cells, (2) cells treated with 1 μM of Compound Selective-1, and (3) cells treated with 5 μM of Compound Selective-1. Y-axis is frequency (%) and X-axis is the number of remaining rhabdomeres.

FIG. 6: Results from Two-Choice Swim Test with R6/2 Mouse. In the R6/2 mouse study described herein, as shown in FIG. 6, animals treated with 5 mg/kg Compound Dual-1 had improved response in the two-choice swim test as compared to vehicle and sertraline treated animals, in a statistically significant manner. FIG. 6 shows the results obtained, from left to right, with WT (i.e., wild-type) vehicle, TO (i.e., transgenic) vehicle, TG 5 mg/kg Dual-1, TG 10 mg/kg Dual-1 and TG 10 mg/kg Sertraline, respectively.

FIG. 7: Longevity Study with R6/2 Mouse. In the R612 mouse study described herein, as shown in FIG. 7, female animals treated with 10 mg/kg Compound Dual-1 survived longer than the other groups of animal, notably the vehicle and sertraline treated animals, in a statistically significant manner.

FIG. 8: Longevity Study with R6/2 Mouse. In the R6/2 mouse study described herein, as shown in FIG. 8, animals (male and female) treated with 10 mg/kg Compound Dual-1 survived longer than the other groups of animal—notably the vehicle and sertraline treated animals in a statistically significant manner.

FIG. 9: Body weight loss in the R6/2 mouse study. The data shown in this graph show that mice treated with Compound Dual-1 at two different doses (5 mg/kg or 10 mg/kg) did not have substantially different weight losses as compared to transgenic animal (“TG”) treated with vehicle or transgenic animal treated with sertraline at 10 mg/kg.

### DETAILED DESCRIPTION OF THE INVENTION

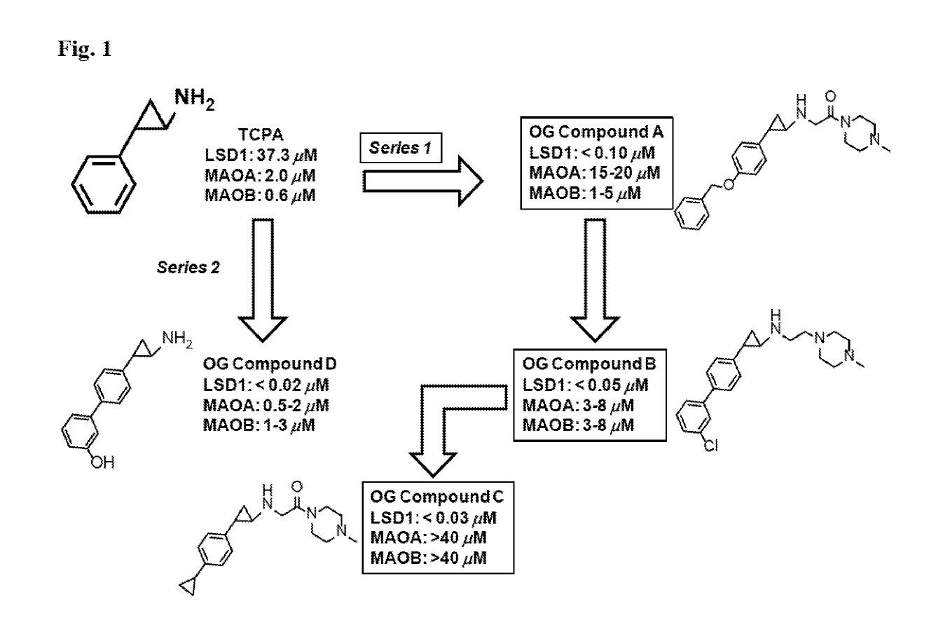

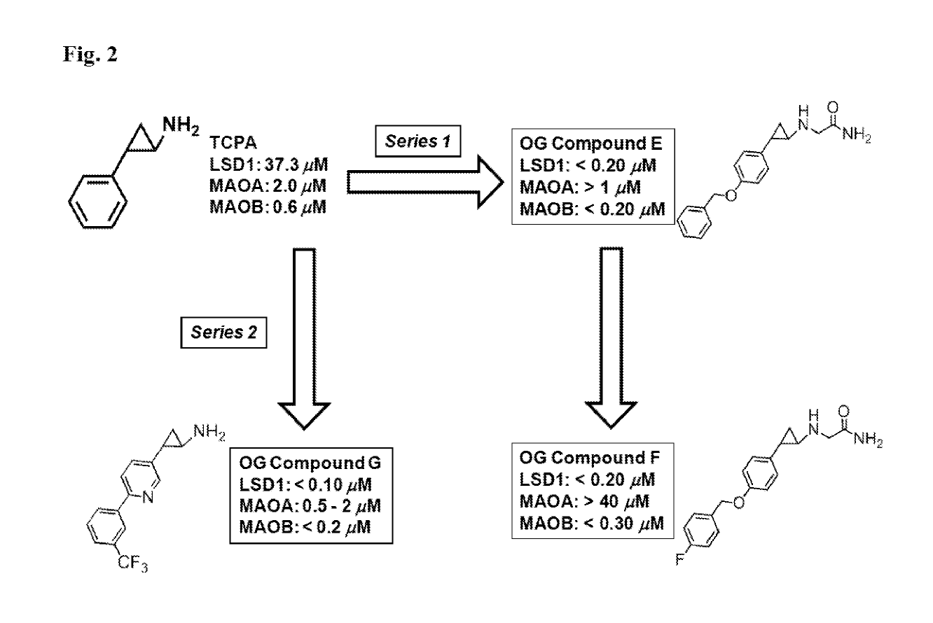

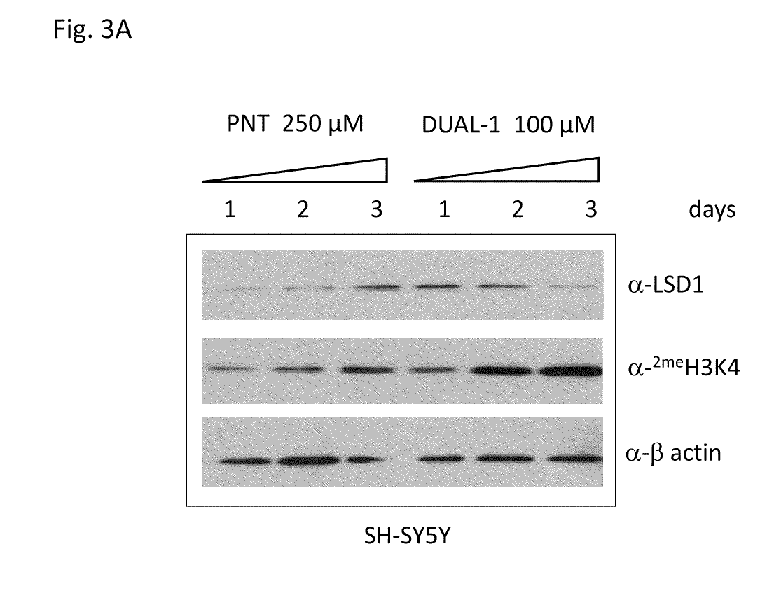

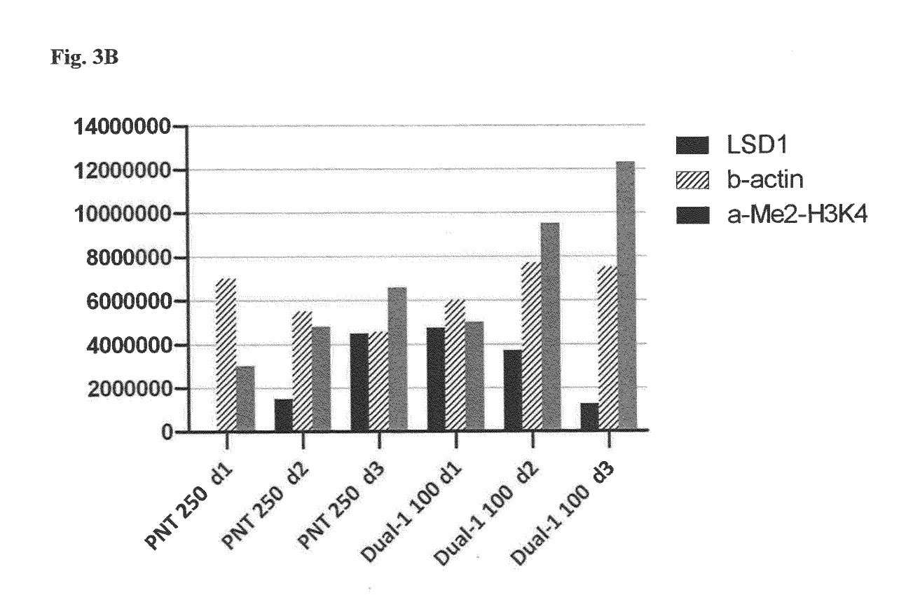

The inventors have surprisingly found that selective potent LSD11 inhibitors and dual inhibitors of LSD1 and MAO-B have therapeutic effects in protein conformation or folding disorder disease models like the R6/2 mouse model or Drosophila models. A medicinal chemistry effort undertaken by some of the inventors resulted in the synthesis and identification of small molecule, potent selective LSD1 inhibitors and potent dual inhibitors of LSD1 and MAO-B. This effort resulted in the identification of a number of compounds having different selectivities for LSD1, MAO-A, and MAO-B. See FIG. 1.

Subsequent studies of some of the optimized compounds in a neural derived cell line indicted that both selective LSD1 inhibitors and dual inhibitors of LSD1 and MAO-B can increase histone methylation levels at the cellular level.

The Huntington drosophila fly line was used to show that treatment of Huntington flies with selective LSD1 and dual LSD1/MAO-B inhibitors were able to rescue eye degeneration phenotype induced by expression of polyQ expanded Huntington.

Studies in the R6/2 mouse showed that (1) inhibitors of LSD1 could be given chronically without gross toxic effects to mammals and that (2) chronic administration of LSD1 inhibitors, particularly dual inhibitors of LSD11 and MAO-B, reduced the decline of some symptoms in of the R6/2 mouse compared to control mice.

Thus, in sum, the inventors have demonstrated that LSD1 inhibitors and dual LSD1/MAO-B inhibitors can be used in chronic treatment regimens suitable for long term treatment in mammals without gross toxicity. Thus, LSD1 and LSD1/MAO-B inhibitors have characteristics suitable for treating protein conformation disorders in a chronic treatment setting e.g., Huntington disease.

### Methods of Treatment or Prevention and Diseases

The patient, subject, or individual, such as the individual in need of treatment or prevention, may be, e.g., a eukaryote, an animal, a vertebrate animal, a mammal, a rodent (e.g., a guinea pig, a hamster, a rat, a mouse), a murine (e.g., a mouse), a canine (e.g., a dog), a feline (e.g., a cat), an equine (e.g., a horse), a primate, a simian (e.g., a monkey or ape), a monkey (e.g., a marmoset, a baboon), an ape (e.g., gorilla, chimpanzee, orangutan, gibbon), or a human. The meaning of the terms “eukaryote,” “animal,” “mammal,” etc., is well known in the art and can, for example, be deduced from Wehner und Gehring (1995; Thieme Verlag). In the context of this invention, it is particularly envisaged that animals are to be treated which are economically, agronomically or scientifically important. Scientifically important organisms include, but are not limited to, mice, rats, rabbits, fruit flies like Drosophila melagonaster and nematodes like Caenorhabditis elegans. Non-limiting examples of agronomically important animals are sheep, cattle and pig, while, for example, cats and dogs may be considered as economically important animals. Preferably, the subject/patient/individual is a mammal; more preferably, the subject/patient/individual is a human.

As used herein, the term “treating a disease or disorder” refers to a slowing of or a reversal of the progress of the disease. Treating a disease or disorder includes treating a symptom and/or reducing the symptoms of the disease.

As used herein, the term “preventing a disease or disorder” refers to a slowing of the disease or of the onset of the disease or the symptoms thereof. Preventing a disease or disorder can include stopping the onset of the disease or symptoms thereof.

As used herein, the term “unit dosage form” refers to a physically discrete unit, such as a capsule or tablet suitable as a unitary dosage for a human patient. Each unit contains a predetermined quantity of an LSD1 inhibitor, which was discovered or believed to produce the desired pharmacokinetic profile which yields the desired therapeutic effect. The dosage unit is composed of an LSD1 inhibitor in association with at least one pharmaceutically acceptable carrier, salt, excipient, or combination thereof.

Preferably, the individual in need of treatment or treatment has a disease associated with a protein conformation disorder or is at risk of having such a disease.

In one aspect, the invention is a method of treating or preventing a cognitive symptom in an individual having a protein conformation disorder comprising identifying a patient in need of such treatment and administering to said individual for a sufficient period of time an amount of an LSD11 inhibitor sufficient to improve the cognitive symptom or reduce the rate of decline of the cognitive symptom thereby treating or preventing said cognitive symptom. In a related aspect, the invention is the use of an LSD1 inhibitor in an amount sufficient to modulate LSD1 activity for treating or preventing cognitive decline in a protein conformation disorder. In a specific aspect, cognitive decline in a protein conformation disorders refers to cognitive decline in a CAG expansion disease. In a more specific aspect, the CAG expansion disorder is Huntington Disease.

In one embodiment of this aspect, the amount of selective LSD1 inhibitor administered is sufficient to modulate or inhibit LSD1 activity while not substantially inhibiting MAO-A activity, thereby avoiding or reducing side-effects associated with administration of MAO-A inhibitors. In a specific aspect of this embodiment, preferably the amount of LSD1 inhibitor administered per day to a human is from about 0.5 mg to about 500 mg per day. More preferably, the amount of LSD1 inhibitor administered per day to a human is from about 0.5 mg to about 200 mg per day or is a pharmaceutical composition formulated in such a way as to deliver this amount of free base equivalent (or free acid equivalent depending on the parent molecule).

In one embodiment of this aspect, the amount of selective LSD1 inhibitor administered is sufficient to modulate or inhibit LSD1 activity while not substantially inhibiting MAO-A activity, thereby avoiding or reducing side-effects associated with administration of MAO-A inhibitors. Preferably, the LSD1 inhibitor is administered or formulated to be administered for five or more days to the individual, more preferably from five days to four years, even more preferably from five days to two years, yet even more preferably for fifteen days to two years, and again yet even more preferably from fifteen days to one year. It is noted that in this context administration for, e.g., five or more days, means an amount sufficient over a time sufficient to cause pharmacologic inhibition of LSD1 over this period of time and this does not necessarily mean administration of compound every day or only once per day. Depending on the PK/ADME properties of the inhibitors, a suitable amount and dosing regimen can be determined by a skilled practitioner in view of this disclosure.

In another aspect, the invention is a method of treating or preventing a motor symptom in an individual having a protein conformation disorder comprising identifying an individual in need of such treatment and administering to said individual for a sufficient period of time an amount of an LSD1 inhibitor sufficient to reduce the rate of decline in said motor symptom thereby treating said motor symptom. In a related aspect, the invention is the use of an LSD1 inhibitor in an amount sufficient to modulate LSD1 activity for treating or preventing a motor symptom in a protein conformation disorder. In a specific aspect, cognitive decline in a protein conformation disorders refers to cognitive decline in a CAG expansion disease. In a more specific aspect, the CAG expansion disorder is Huntington Disease.

In one embodiment of this aspect, the amount of selective LSD1 inhibitor administered is sufficient to modulate or inhibit LSD1 activity while not substantially inhibiting MAO-A activity, thereby avoiding or reducing side-effects associated with administration of MAO-A inhibitors. In a specific aspect of this embodiment, preferably the amount of LSD1 inhibitor administered per day to a human is from about 0.5 mg to about 500 mg per day. More preferably the amount of LSD1 inhibitor administered per day to a human is from about 0.5 mg to about 200 mg per day or is a pharmaceutical composition formulated in such a way as to deliver this amount of free base equivalent (or free acid equivalent depending on the parent molecule). Preferably, the LSD1 inhibitor is administered or formulated to be administered for five or more days to the individual, more preferably from five days to four years, even more preferably, from five days to two years, yet even more preferably for fifteen days to two years, and again yet even more preferably from fifteen days to one year. It is noted that in this context administration for, e.g., five or more days, means an amount sufficient over a time sufficient to cause pharmacologic inhibition of LSD1 over this period of time and this does not necessarily mean administration of compound every day or only once per day. Depending on the PK/ADME properties of the inhibitors, a suitable amount and dosing regimen can be determined by a skilled practitioner in view of this disclosure.

In another aspect, the invention is a method of increasing longevity in an individual having a protein conformation disorder comprising identifying an individual in need of such treatment and administering to said individual for a sufficient period of time an amount of an LSD1 sufficient to increase longevity. In a related aspect, the invention is the use of an LSD1 inhibitor in an amount sufficient to modulate LSD1 activity for treating or preventing decreased longevity associated with a protein conformation disorder. In a specific aspect, cognitive decline in a protein conformation disorders refers to cognitive decline in a CAG expansion disease. In a more specific aspect, the CAG expansion disorder is Huntington Disease.

In one embodiment of this aspect, the amount of selective LSD1 inhibitor administered is sufficient to modulate or inhibit LSD1 activity while not substantially inhibiting MAO-A activity, thereby avoiding or reducing side-effects associated with administration of MAO-A inhibitors. In a specific aspect of this embodiment, preferably the amount of LSD1 inhibitor administered per day to a human is from about 0.5 mg to about 500 mg per day. More preferably, the amount of LSD1 inhibitor administered per day to a human is from about 0.5 mg to about 200 mg per day or is a pharmaceutical composition formulated in such a way as to deliver this amount of free base equivalent (or free acid equivalent depending on the parent molecule). Preferably, the LSD1 inhibitor is administered or formulated to be administered for five or more days to the individual, more preferably from five days to four years, even more preferably from five days to two years, yet even more preferably for fifteen days to two years, and again yet even more preferably from fifteen days to one year. It is noted that in this context administration for, e.g., five or more days, means an amount sufficient over a time sufficient to cause pharmacologic inhibition of LSD1 over this period of time and this does not necessarily mean administration of compound every day or only once per day. Depending on the PK/ADME properties of the inhibitors, a suitable amount and dosing regimen can be determined by a skilled practitioner in view of this disclosure.

In one aspect, the invention is a method of treating or preventing a cognitive symptom in an individual having a protein conformation disorder comprising identifying a patient in need of such treatment and administering to said individual for a sufficient period of time an amount of a dual LSD1/MAO-B inhibitor sufficient to improve the cognitive symptom or reduce the rate of decline of the cognitive symptom thereby treating or preventing said cognitive symptom. In a related aspect, the invention is the use of a dual LSD1/MAO-B inhibitor in an amount sufficient to modulate LSD1 activity for treating or preventing cognitive decline in a protein conformation disorder. In a specific aspect, cognitive decline in a protein conformation disorders refers to cognitive decline in a CAG expansion disease. In a more specific aspect, the CAG expansion disorder is Huntington Disease.

In one embodiment of this aspect, the amount of selective LSD1 inhibitor administered is sufficient to modulate or inhibit LSD1 and MAO-B activity while not substantially inhibiting MAO-A activity, thereby avoiding or reducing side-effects associated with administration of MAO-A inhibitors. In a specific aspect of this embodiment, preferably the amount of LSD1/MAO-B inhibitor administered per day to a human is from about 0.5 mg to about 500 mg per day. More preferably the amount of LSD1/MAO-B inhibitor administered per day to a human is from about 0.5 mg to about 200 mg per day or is a pharmaceutical composition formulated in such a way as to deliver this amount of free base equivalent (or free acid equivalent depending on the parent molecule).

In one embodiment of this aspect, the amount of selective LSD1 inhibitor administered is sufficient to modulate or inhibit LSD11/MAO-B activity while not substantially inhibiting MAO-A activity, thereby avoiding or reducing side-effects associated with administration of MAO-A inhibitors. Preferably, the dual LSD1/MAO-B inhibitor is administered or formulated to be administered for five or more days to the individual, more preferably from five days to four years, even more preferably from five days to two years, yet even more preferably for fifteen days to two years, and again yet even more preferably from fifteen days to one year. It is noted that in this context administration for, e.g., five or more days, means an amount sufficient over a time sufficient to cause pharmacologic inhibition of LSD1 and MAO-B over this period of time and this does not necessarily mean administration of compound every day or only once per day. Depending on the PK/ADME properties of the inhibitors, a suitable amount and dosing regimen can be determined by a skilled practitioner in view of this disclosure.

In another aspect, the invention is a method of treating or preventing a motor symptom in an individual having a protein conformation disorder comprising identifying an individual in need of such treatment and administering to said individual for a sufficient period of time an amount of a dual LSD1/MAO-B inhibitor sufficient to reduce the rate of decline in said motor symptom thereby treating said motor symptom. In a related aspect, the invention is the use of a dual LSD1/MAO-B inhibitor in an amount sufficient to modulate LSD1 and MAO-B activity for treating or preventing a motor symptom in a protein conformation disorder. In a specific aspect, cognitive decline in a protein conformation disorders refers to cognitive decline in a CAG expansion disease. In a more specific aspect, the CAG expansion disorder is Huntington Disease.

In one embodiment of this aspect, the amount of the dual LSD1/MAO-B inhibitor administered is sufficient to modulate or inhibit LSD and MAO-B activity while not substantially inhibiting MAO-A activity, thereby avoiding or reducing side-effects associated with administration of MAO-A inhibitors. In a specific aspect of this embodiment, preferably the amount of LSD1 inhibitor administered per day to a human is from about 0.5 mg to about 500 mg per day. More preferably, the amount of the dual LSD1/MAO-B inhibitor administered per day to a human is from about 0.5 mg to about 200 mg per day or is a pharmaceutical composition formulated in such a way as to deliver this amount of free base equivalent (or free acid equivalent depending on the parent molecule). Preferably, the dual LSD1/MAO-B inhibitor is administered or formulated to be administered for five or more days to the individual, more preferably from five days to four years, even more preferably from five days to two years, yet even more preferably for fifteen days to two years, and again yet even more preferably from fifteen days to one year. It is noted that in this context administration for, e.g., five or more days, means an amount sufficient over a time sufficient to cause pharmacologic inhibition of LSD1 and MAO-B over this period of time and this does not necessarily mean administration of compound every day or only once per day. Depending on the PK/ADME properties of the inhibitors, a suitable amount and dosing regimen can be determined by a skilled practitioner in view of this disclosure.

In another aspect, the invention is a method of increasing longevity in an individual having a protein conformation disorder comprising identifying an individual in need of such treatment and administering to said individual for a sufficient period of time an amount of a dual LSD1/MAO-B sufficient to increase longevity. In a related aspect, the invention is the use of a dual LSD1/MAO-B inhibitor in an amount sufficient to modulate LSD1 and MAO-B activity for treating or preventing decreased longevity associated with a protein conformation disorder. In a specific aspect, cognitive decline in a protein conformation disorders refers to cognitive decline in a CAG expansion disease. In a more specific aspect, the CAG expansion disorder is Huntington Disease.

In one embodiment of this aspect, the amount of dual LSD1/MAO-B inhibitor administered is sufficient to modulate or inhibit LSD1 and MAO-B activity while not substantially inhibiting MAO-A activity, thereby avoiding or reducing side-effects associated with administration of MAO-A inhibitors. In a specific aspect of this embodiment, preferably the amount of the dual LSD1/MAO-B inhibitor administered per day to a human is from about 0.5 mg to about 500 mg per day. More preferably the amount of LSD1 inhibitor administered per day to a human is from about 0.5 mg to about 200 mg per day or is a pharmaceutical composition formulated in such a way as to deliver this amount of free base equivalent (or free acid equivalent depending on the parent molecule). Preferably, the LSD1/MAO-B inhibitor is administered or formulated to be administered for five or more days to the individual, more preferably from five days to four years, even more preferably from five days to two years, yet even more preferably for fifteen days to two years, and again yet even more preferably from fifteen days to one year. It is noted that in this context administration for, e.g., five or more days, means an amount sufficient over a time sufficient to cause pharmacologic inhibition of LSD1 and MAO-B over this period of time and this does not necessarily mean administration of compound every day or only once per day. Depending on the PK/ADME properties of the inhibitors, a suitable amount and dosing regimen can be determined by a skilled practitioner in view of this disclosure.

In one aspect, the protein conformation disorder is a CAG expansion disorder.

In again another aspect, the protein conformation disorder is Alzheimer Disease.

In still another aspect, the protein conformation disorder is Parkinson Disease.

In one aspect, the CAG repeat disorder is Huntington disease, Kennedy Disease, Spinocerebellar Ataxia 1, Spinocerebellar Ataxia 2, Spinocerebellar Ataxia 3, Spinocerebellar Ataxia 6, Spinocerebellar Ataxia 7, or Spinocerebellar Ataxia 17.

In one aspect, the CAG repeat disorder is Huntington disease.

In one aspect, the sufficient period of time for administering the LSD1 or LSD1/MAO-B dual inhibitors is from five or more days to the individual, more preferably from five days to four years, even more preferably from five days to two years, yet even more preferably for fifteen days to two years, and again yet even more preferably from fifteen days to one year. In one aspect, the LSD1 or LSD1/MAO-B inhibitor is administered daily in amount sufficient to yield a Cmax above the IC50 value for the LSD1 inhibitor. The Cmax can be determined using any standard assay known in the art.

The invention also relates to an LSD1 inhibitor for use in any of the above-described methods.

Accordingly, the invention relates to an LSD1 inhibitor (or a pharmaceutical composition comprising an LSD1 inhibitor and a pharmaceutically acceptable carrier) for use in treating or preventing a protein conformation disorder. The invention also relates to an LSD1 inhibitor (or a pharmaceutical composition comprising an LSD1 inhibitor and a pharmaceutically acceptable carrier) for use in treating or preventing a cognitive symptom or cognitive decline in an individual (preferably a mammal; more preferably a human) having a protein conformation disorder. Likewise, the invention encompasses a an LSD1 inhibitor (or a pharmaceutical composition comprising an LSD1 inhibitor and a pharmaceutically acceptable carrier) for use in improving a cognitive symptom in an individual (preferably a mammal; more preferably a human) having a protein conformation disorder or for use in reducing the rate of decline of a cognitive symptom in an individual (preferably a mammal; more preferably a human) having a protein conformation disorder. The invention also relates to an LSD1 inhibitor (or a pharmaceutical composition comprising an LSD1 inhibitor and a pharmaceutically acceptable carrier) for use in treating or preventing a motor symptom in an individual (preferably a mammal; more preferably a human) having a protein conformation disorder. Moreover, the invention encompasses an LSD1 inhibitor (or a pharmaceutical composition comprising an LSD1 inhibitor and a pharmaceutically acceptable carrier) for use in increasing longevity in an individual (preferably a mammal; more preferably a human) having a protein conformation disorder or for use in treating or preventing decreased longevity associated with a protein conformation disorder.

The protein conformation disorder may, e.g., be a CAG expansion disease (or CAG expansion disorder or CAG repeat disorder), such as Huntington Disease, Kennedy Disease, Spinocerebellar Ataxia 1. Spinocerebellar Ataxia 2, Spinocerebellar Ataxia 3, Spinocerebellar Ataxia 6, Spinocerebellar Ataxia 7, or Spinocerebellar Ataxia 17. The protein conformation disorder may also be Alzheimer Disease or Parkinson Disease. The present invention particularly relates to the treatment or prevention of Huntington Disease using an LSD1 inhibitor.

### Compounds, Formulation, Routes of Administration, and PK/TOX

The selective LSD1 inhibitors and dual LSD1/MAO-B inhibitors for use in the invention can be synthesized by a number of techniques. Examples of selective LSD1 and LSD1/MAO-B dual inhibitors are given in, e.g., WO2010/043721 (PCT/EP2009/063685), WO2010/084160 (PCT/EP2010/050697), WO2011/035941 (PCT/EP2010/055131), WO2011/042217 (PCT/EP2010/055103), PCT/EP2011/062947, PCT/EP2011/056279, PCT/EP2011/062949, and EP application numbers EP10171345 (EP EP10171345.1) and EP10187039.2, all of which are explicitly incorporated herein by reference in their entireties to the extent they are not inconsistent with the instant disclosure.

Other examples of LSD1 inhibitors are, e.g., phenelzine or pargyline or a derivative or analog thereof. Derivatives and analogs of phenelzine and pargyline include, but are not limited to, compounds where the phenyl group of the parent compound is replaced with a heteroaryl or optionally substituted cyclic group or the phenyl group of the parent compound is optionally substituted with a cyclic group and have the selective LSD1 or dual LSD1/MAO-B inhibitory activity as described herein.

The LSD1 inhibitor or selective LSD1 inhibitor or dual LSD1/MAO-B inhibitor to be used in accordance with the present invention is preferably a 2-cyclylcyclopropan-1-amine compound, a phenelzine compound or a propargylamine compound, and is more preferably a 2-cyclylcyclopropan-1-amine compound. Said 2-cyclylcyclopropan-1-amine compound is preferably a 2-arylcyclopropan-1-amine compound or a 2-heteroarylcyclopropan-1-amine compound, more preferably a 2-phenylcyclopropan-1-amine compound or a 2-pyridinylcyclopropan-1-amine compound.

It is particularly preferred that the LSD1 inhibitor or selective LSD1 inhibitor or dual LSD1/MAO-B inhibitor is a 2-cyclylcyclopropan-1-amine compound which is a compound of the following formula (I) or an enantiomer, a diastereomer or a racemic mixture thereof, or a pharmaceutically acceptable salt or solvate thereof:

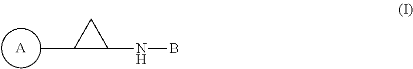

A is cyclyl optionally having 1, 2, 3 or 4 substituents A′. Preferably, said cyclyl is aryl or heteroaryl. Said aryl is preferably phenyl. Said heteroaryl is preferably selected from pyridinyl, pyrimidinyl, thiophenyl, benzothiophenyl, pyrrolyl, indolyl, furanyl or thiazolyl, more preferably said heteroaryl is selected from pyridinyl, pyrimidinyl or thiophenyl, and even more preferably said heteroaryl is pyridinyl (in particular, pyridin-2-yl or pyridin-3-yl).
It is preferred that said cyclyl (or said aryl or said heteroaryl, or any of the above-mentioned specific aryl or heteroaryl groups) is unsubstituted or has 1 or 2 substituents A′, and it is more preferred that said cyclyl (or said aryl or said heteroaryl, or any of the above-mentioned specific aryl or heteroaryl groups) is unsubstituted or has 1 substituent A′.
Said substituent(s) A′ is/are each independently selected from -L¹-cyclyl (e.g., -L¹-aryl, -L¹-cycloalkyl or -L¹-heterocyclyl), alkyl, alkenyl, alkynyl, alkoxy, amino, amido (e.g., —CO—NH₂), —CH₂—CO—NH₂, alkylamino, hydroxyl, nitro, halo, haloalkyl, haloalkoxy, cyano, sulfonyl, sulfinyl, sulfonamide, acyl, carboxyl, carbamate or urea, wherein the cyclyl moiety comprised in said -L¹-cyclyl is optionally further substituted with one or more (e.g., 1, 2 or 3) groups independently selected from halo, haloalkyl, haloalkoxy, aryl, arylalkoxy, aryloxy, arylalkyl, alkyl, alkenyl, alkynyl, alkoxy, amino, amido (e.g., —CO—NH₂), alkylamino, hydroxyl, nitro, —CH₂—CO—NH₂, heteroaryl, heteroarylalkoxy, heteroaryloxy, heteroarylalkyl, cyano, sulfonyl, sulfinyl, sulfonamide, acyl, carboxyl, carbamate or urea, preferably selected from halo, haloalkyl or cyano. It is preferred that the cyclyl moiety comprised in said -L¹-cyclyl is unsubstituted or is substituted with one of the above groups (including, e.g., one of the preferred groups halo, haloalkyl or cyano), and it is more preferred that the cyclyl moiety is unsubstituted. Said -L¹-cyclyl is preferably -L¹-aryl, -L¹-cycloalkyl or -L¹-heterocyclyl (e.g., -L¹-heteroaryl or -L¹-heterocycloalkyl), more preferably -L‘-aryl or -L’-heteroaryl, even more preferably -L¹-aryl, even more preferably -L¹-phenyl.
Each L¹ is independently selected from a covalent bond, —(CH₂)₁₋₆—, —(CH₂)₀₋₃—O—(CH₂)₀₋₃—, —(CH₂)₀₋₃—NH—(CH₂)₀₋₃- or —(CH₂)₀₋₃—S—(CH₂)₀₋₃—, preferably from a covalent bond, —(CH₂)₁₋₃—, —O—(CH₂)₀₋₃- or —NH—(CH₂)₀₋₃—, more preferably from a covalent bond, —CH₂—, —O—, —O—CH₂—, —O—(CH₂)₂—, —NH— or —NH—CH₂—, even more preferably from a covalent bond, —CH₂— or —O—CH₂—. It is furthermore preferred that the aforementioned groups L¹ (connecting the moiety A to the cyclyl moiety comprised in -L¹-cyclyl) are in the specific orientation indicated above (accordingly, the group “—O—CH₂—” as an example for L¹ is preferably in the orientation ( . . . )-A-O—CH₂-cyclyl).
Preferably, said substituent(s) A′ is/are each independently selected from -L¹-aryl, -L¹-cycloalkyl, -L¹-heteroaryl or -L¹-heterocycloalkyl, wherein said aryl, said cycloalkyl, said heteroaryl or said heterocycloalkyl is optionally substituted with halo (e.g., —F or —Cl), haloalkyl (e.g., —CF₃) or cyano. More preferably, said substituent(s) A′ is/are each independently -L¹-aryl (e.g., -L¹-phenyl), wherein the aryl moiety in said -L¹-aryl (or the phenyl moiety in said -L¹-phenyl) is optionally substituted with halo (e.g., —F or —Cl), haloalkyl (e.g., —CF₃) or cyano. Even more preferably, said substituent(s) A′ is/are each independently phenyl, —CH₂-phenyl, —O—CH₂-phenyl or —O—(CH₂)₂-phenyl, wherein said phenyl or the phenyl moiety in said —CH₂-phenyl, said —O—CH₂-phenyl or said —O—(CH₂)₂-phenyl is optionally substituted with halo (e.g., —F or —Cl), haloalkyl (e.g., —CF₃) or cyano. Even more preferably, said substituent(s) A′ is/are each independently phenyl, —CH₂-phenyl, or —O—CH₂-phenyl, wherein said phenyl or the phenyl moiety in said —CH₂-phenyl or said —O—CH₂-phenyl is optionally substituted with halo (e.g., —F or —Cl) or haloalkyl (e.g., —CF₃).
It is particularly preferred that A is aryl (preferably phenyl) or heteroaryl (preferably pyridinyl), which aryl or heteroaryl optionally has one substituent A′ selected from -L¹-aryl, -L¹-cycloalkyl, -L¹-heteroaryl or -L¹-heterocycloalkyl (wherein the aryl moiety in said -L¹-aryl, the cycloalkyl moiety in said -L¹-cycloalkyl, the heteroaryl moiety in said -L¹-heteroaryl or the heterocycloalkyl moiety in said -L¹-heterocycloalkyl may be substituted with halo (e.g., —F or —Cl), haloalkyl (e.g., —CF₃) or cyano), preferably selected from phenyl, —CH₂-phenyl or —O—CH₂-phenyl (wherein said phenyl, the phenyl moiety in said —CH₂-phenyl or the phenyl moiety in said —O—CH₂-phenyl may be substituted with halo (e.g., —F or —Cl) or haloalkyl (e.g., —CF₃)).
B is —H, -L²-CO—NH₂ or -L²-cyclyl, wherein the cyclyl moiety in said -L²-cyclyl is optionally substituted with one or more (e.g., one, two or three) groups independently selected from halo, haloalkyl, haloalkoxy, haloaryl, aryl, arylalkoxy, aryloxy, arylalkyl, alkyl, alkenyl, alkynyl, alkoxy, amino, amido (e.g., —CO—NH₂), alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate or urea, preferably selected from halo, alkyl, alkoxy, haloalkyl, haloalkoxy, cyano, hydroxyl, amino, alkylamino, aminoalkyl, amido (e.g., —CO—NH₂), —CH₂—CO—NH₂, or sulfonamide.
It is preferred that the cyclyl moiety in said -L²-cyclyl is unsubstituted or is substituted with one group selected from halo, haloalkyl, haloalkoxy, haloaryl, aryl, arylalkoxy, aryloxy, arylalkyl, alkyl, alkenyl, alkynyl, alkoxy, amino, amido (e.g., —CO—NH₂), alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate or urea, preferably selected from halo, alkyl, alkoxy, haloalkyl, haloalkoxy, cyano, hydroxyl, amino, alkylamino, aminoalkyl, amido (e.g., —CO—NH₂), —CH₂—CO—NH₂, or sulfonamide.
The cyclyl moiety in said -L¹-cyclyl, which may be substituted as defined and described above, is preferably selected from aryl, cycloalkyl or heterocyclyl (e.g., heteroaryl or heterocycloalkyl), more preferably from heterocyclyl, even more preferably from heteroaryl or heterocycloalkyl. Said heteroaryl is preferably selected from oxadiazolyl, thiazolyl or pyrimidinyl. Said heterocycloalkyl is preferably selected from pyrrolidinyl, piperidinyl, piperazinyl, N-methylpiperazinyl or morpholinyl.
L² is C₁₋₁₂ alkylene which is optionally interrupted by one or more (e.g., one, two, three or four) groups independently selected from —O—, —S—, —NH—, —N(alkyl)-, —CO—, —CO—NH— or —CO—N(alkyl)-, or L² is a covalent bond. Preferably, L² is —CH₂—(C₁₋₆ alkylene), —CH₂—CO— or a covalent bond, wherein the alkylene moiety in said —CH₂—(C₁₋₆ alkylene) is optionally interrupted by one or more (e.g., one, two or three) groups independently selected from —O—, —S—, —NH—, —N(alkyl)-, —CO—, —CO—NH—, —CO—N(alkyl)-. More preferably, L² is —(CH₂)₁₋₄—, —CH₂—CO— or a covalent bond. Even more preferably, L² is —CH₂—, —(CH₂)₂—, —CH₂—CO— or a covalent bond.
Preferably, B is —H, —(CH₂)₁₋₄—CO—NH₂, —(CH₂)₀₋₅-heteroaryl, —(CH₂)₀₋₅-heterocycloalkyl or —(CH₂)₁₋₅—CO-heterocycloalkyl, wherein the heteroaryl moiety comprised in said —(CH₂)₀₋₅-heteroaryl or the heterocycloalkyl moiety comprised in said —(CH₂)₀₋₅-heterocycloalkyl or in said —(CH₂)₁₋₅—CO-heterocycloalkyl is optionally substituted with one group selected from halo, alkyl, alkoxy, haloalkyl, haloalkoxy, cyano, hydroxyl, amino, alkylamino, aminoalkyl, amido (e.g., —CO—NH₂), —CH₂—CO—NH₂, or sulfonamide.
In a particularly preferred embodiment, B is —H. In a further particularly preferred embodiment, B is —(CH₂)₁₋₄—CO—NH₂, more preferably —CH₂—CO—NH₂. In a further particularly preferred embodiment, B is —(CH₂)₀₋₅-heteroaryl, wherein the heteroaryl moiety comprised in said —(CH₂)₀₋₅-heteroaryl is preferably selected from oxadiazolyl, thiazolyl or pyrimidinyl and, furthermore, is optionally substituted with one group selected from halo, alkyl, alkoxy, haloalkyl, haloalkoxy, cyano, hydroxyl, amino, alkylamino, aminoalkyl, amido (e.g., —CO—NH₂), —CH₂—CO—NH₂, or sulfonamide. In a further particularly preferred embodiment, B is —(CH₂)₀₋₅-heterocycloalkyl, wherein the heterocycloalkyl moiety comprised in said —(CH₂)₀₋₅-heterocycloalkyl is preferably selected from pyrrolidinyl, piperidinyl, piperazinyl, N-methylpiperazinyl or morpholinyl and, furthermore, is optionally substituted with one group selected from halo, alkyl, alkoxy, haloalkyl, haloalkoxy, cyano, hydroxyl, amino, alkylamino, aminoalkyl, amido (e.g., —CO—NH₂), —CH₂—CO—NH₂, or sulfonamide. In a further particularly preferred embodiment, B is —CH₂-oxadiazolyl, wherein the oxadiazolyl moiety comprised in said —CH₂-oxadiazolyl is optionally substituted with one group selected from halo, alkyl, alkoxy, haloalkyl, haloalkoxy, cyano, hydroxyl, amino, alkylamino or aminoalkyl (accordingly, B may, for example, be aminooxadiazolylmethyl, such as 2-amino-1,3,4-oxadiazol-5-ylmethyl or 3-amino-1,2,4-oxadiazol-5-ylmethyl). In a further particularly preferred embodiment, B is —(CH₂)₁₋₅—CO-heterocycloalkyl, wherein the heterocycloalkyl moiety comprised in said —(CH₂)₁₋₅—CO-heterocycloalkyl is preferably selected from pyrrolidinyl, piperidinyl, piperazinyl, N-methylpiperazinyl or morpholinyl and, furthermore, is optionally substituted with one group selected from halo, alkyl, alkoxy, haloalkyl, haloalkoxy, cyano, hydroxyl, amino, alkylamino, aminoalkyl, amido (e.g., —CO—NH₂), —CH₂—CO—NH₂, or sulfonamide.
The substituents on the cyclopropane ring, i.e. the groups -(A) and —NH—B, are preferably in trans configuration. In that case, the 2-cyclylcyclopropan-1-amine compound of formula (I) may have the configuration (1R,2S) or the configuration (1S,2R) at the cyclopropane ring carbon atoms. The present invention specifically relates to the (1R,2S) stereoisomer of the 2-cyclylcyclopropan-1-amine compound of formula (I). The invention also specifically relates to the (1S,2R) stereoisomer of the 2-cyclylcyclopropan-1-amine compound of formula (I).

In one embodiment, the LSD1 inhibitor or selective LSD1 inhibitor or dual LSD1/MAO-B inhibitor to be used in accordance with the present invention is a 2-cyclylcyclopropan-1-amine compound which is a compound of the following formula (II) or a pharmaceutically acceptable salt thereof:

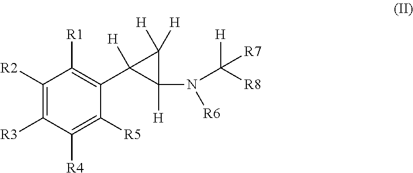

In formula (II), each of R1-R5 is optionally substituted and independently chosen from —H, halo, alkyl, alkoxy, cycloalkoxy, haloalkyl, haloalkoxy, -L-aryl, -L-heteroaryl, -L-heterocyclyl, -L-carbocycle, acylamino, acyloxy, alkylthio, cycloalkylthio, alkynyl, amino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamide, thiocarbonyl, thiocyanato, trihalomethanesulfonamnido, O-carbamyl, N-carbamyl, O-thiocarbamyl, N-thiocarbamyl, and C-amido;
R6 is chosen from —H and alkyl;
R7 is chosen from —H, alkyl, and cycloalkyl;
R8 is chosen from —C(═O)NRₓR_{y} and —C(═O)R₂;
Rₓ when present is chosen from —H, alkyl, alkynyl, alkenyl, -L-carbocycle, -L-aryl, -L-heterocyclyl, all of which are optionally substituted;
R_{y} when present is chosen from —H, alkyl, alkynyl, alkenyl, -L-carbocycle, -L-aryl, -L-heterocyclyl, all of which are optionally substituted;
R_{z} when present is chosen from —H, alkoxy, -L-carbocyclic, -L-heterocyclic, -L-aryl, wherein the aryl, heterocyclyl, or carbocycle is optionally substituted;
each L can be saturated, partially saturated, or unsaturated, and is independently chosen from —(CH₂)ₙ—(CH₂)ₙ—, —(CH₂)ₙC(═O)(CH₂)ₙ—, —(CH₂)ₙC(═O)NH(CH₂)ₙ—, —(CH₂)ₙNHC(═O)O(CH₂)ₙ—, —(CH₂)ₙNHC(═O)NH(CH₂)ₙ—, —(CH₂)ₙNHC(═S)S(CH₂)ₙ—, —(CH₂)ₙOC(═O)S(CH₂)ₙ—, —(CH₂)ₙNH(CH₂)ₙ—, —(CH₂)ₙO(CH₂)ₙ—, —(CH₂)ₙS(CH₂)ₙ—, and —(CH₂)ₙNHC(═S)NH(CH₂)ₙ—, where each n is independently chosen from 0, 1, 2, 3, 4, 5, 6, 7, and 8, wherein optionally substituted refers to zero or 1 to 4 optional substituents independently chosen from acylamino, acyloxy, alkenyl, alkoxy, cycloalkoxy, alkyl, alkylthio, cycloalkylthio, alkynyl, amino, aryl, arylalkyl, arylalkenyl, arylalkynyl, arylalkoxy, aryloxy, arylthio, hoteroarylthio, carbocyclyl, cyano, cyanato, halo, haloalkyl, haloaryl, hydroxyl, heteroaryl, heteroaryloxy, heterocyclyl, heteroarylalkoxy, isocyanato, isothiocyanato, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido.

In a further embodiment, the LSD1 inhibitor or selective LSD1 inhibitor or dual LSD1/MAO-B inhibitor to be used in accordance with the invention is a 2-cyclylcyclopropan-1-amine compound which is a compound of the following formula (III) or a pharmaceutically acceptable salt thereof:

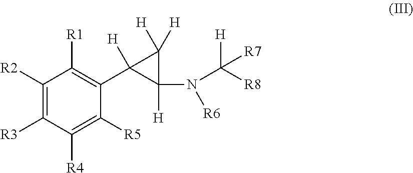

In formula (III), each of R1-R5 is independently chosen from —H, halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamido, thiocarbonyl, thiocyanato, trihalomethanesulfonamido, O-carbamyl, N-carbamyl, O-thiocarbamyl, N-thiocarbamyl, and C-amido;
R6 is chosen from —H and alkyl;
R7 is chosen from —H, alkyl, and cycloalkyl;
R8 is a -L-heterocyclyl wherein the ring or ring system of said -L-heterocyclyl has from 0-3 substituents chosen from halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamido, thiocarbonyl, thiocyanato, trihalomethanesulfonamido, O-carbamyl, N-carbamyl, O-thiocarbamyl, N-thiocarbamyl, and C-amido; or
R8 is -L-aryl wherein the ring or ring system of said -L-aryl has from 1-3 substituents chosen from halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamido, thiocarbonyl, thiocyanato, trihalomethanesulfonamido, O-carbamyl, N-carbamyl, O-thiocarbamyl, N-thiocarbamyl, and C-amido;
each L is independently chosen from —(CH₂)ₙ—(CH₂)ₙ—, —(CH₂)ₙNH(CH₂)ₙ—, —(CH₂)ₙO(CH₂)ₙ—, and —(CH₂)ₙS(CH₂)ₙ—, and where each n is independently chosen from 0, 1, 2, and 3.

In a further embodiment, the LSD1 inhibitor or selective LSD1 inhibitor or dual LSD11/MAO-B inhibitor to be used in accordance with the invention is a 2-cyclylcyclopropan-1-amine compound which is a compound of the following formula (IV) or an enantiomer, diastereomer, or mixture thereof, or a pharmaceutically acceptable salt or solvate thereof:

(A′)x-(A)-(B)—(Z)-(L)-(D) (IV)

In formula (IV), (A) is heteroaryl or aryl;
each (A′), if present, is independently chosen from aryl, arylalkoxy, arylalkyl, heterocyclyl, aryloxy, halo, alkoxy, haloalkyl, cycloalkyl, haloalkoxy, and cyano, wherein each (A′) is substituted with 0, 1, 2, or 3 substituents independently chosen from halo, haloalkyl, aryl, arylalkoxy, alkyl, alkoxy, cyano, sulfonyl, amido, and sulfinyl;

### X is 0, 1, 2, or 3;

(B) is a cyclopropyl ring, wherein (A) and (Z) are covalently bonded to different carbon atoms of (B);

### (Z) is —NH—;

(L) is chosen from —CH₂CH₂—, —CH₂CH₂CH₂—, and —CH₂CH₂CH₂CH₂—; and
(D) is chosen from —N(—R1)-R2, —O—R3, and —S—R3, wherein:
R1 and R2 are mutually linked to form a heterocyclic ring together with the nitrogen atom that R1 and R2 are attached to, wherein said heterocyclic ring has 0, 1, 2, or 3 substituents independently chosen from —NH₂, —NH(C₁-C₆ alkyl), —N(C₁-C₆ alkyl)(C₁-C₆ alkyl), alkyl, halo, cyano, alkoxy, haloalkyl, and haloalkoxy, or
R1 and R2 are independently chosen from —H, alkyl, cycloalkyl, haloalkyl, and heterocyclyl, wherein the sum of substituents on R1 and R2 together is 0, 1, 2, or 3, and the substituents are independently chosen from —NH₂, —NH(C₁-C₆ alkyl), —N(C₁-C₆ alkyl)(C₁-C₆ alkyl), and fluoro; and
R3 is chosen from —H, alkyl, cycloalkyl, haloalkyl, and heterocyclyl, wherein R3 has 0, 1, 2, or 3 substituents independently chosen from —NH₂, —NH(C₁-C₆ alkyl), —N(C₁-C₆ alkyl)(C₁-C₆ alkyl), and fluoro;
with the proviso that the following compounds are excluded:
N1-[(trans)-2-phenylcyclopropyl]-N2-undecyl-rel-1,2-ethanediamine;
N1-[(trans)-2-phenylcyclopropyl]-N2-tricyclo[3.3.1.13,7]dec-2-yl-rel-1,2-ethanediamine;
N1-cyclooctyl-N2-[(trans)-2-phenylcycopropyl]-rel-1,2-ethanediamine;
N1,N1-dimethyl-N2-(2-phenylcyclopropyl)-1,3-propanediamine;
N1,N1-dimethyl-N2-(2-phenylcyclopropyl)-1,2-ethanediamine; and
trans-1-phenyl-2-[(2-hydroxyethyl)amino]cyclopropane.

In a further embodiment, the LSD1 inhibitor or selective LSD1 inhibitor or dual LSD/MAO-B inhibitor to be used in accordance with the invention is a 2-cyclylcyclopropan-1-amine compound which is a compound of the following formula (V) or a pharmaceutically acceptable salt or solvate thereof:

(A′)x-(A)-(B)—(Z)-(L)-C(═O)NH₂ (V)

In formula (V), (A) is heteroaryl or aryl;
each (A′), if present, is independently chosen from aryl, arylalkoxy, arylalkyl, heterocyclyl, aryloxy, halo, alkoxy, haloalkyl, cycloalkyl, haloalkoxy, and cyano, wherein each (A′) is substituted with 0, 1, 2 or 3 substituents independently chosen from halo, haloalkyl, aryl, arylalkoxy, alkyl, alkoxy, cyano, sulfonyl, sulfinyl, and carboxamide;

### X is 0, 1, 2, or 3;

(B) is a cyclopropyl ring, wherein (A) and (Z) are covalently bonded to different carbon atoms of (B);

### (Z) is —NH—; and

(L) is —(CH₂)ₘCR₁R₂—, wherein m is 0, 1, 2, 3, 4, 5, or 6, and wherein R₁ and R₂ are each independently hydrogen or C₁-C₆ alkyl;
provided that, if (L) is —CH₂— or —CH(CH₃)—, then X is not 0.

In a further embodiment, the LSD1 inhibitor or selective LSD1 inhibitor or dual LSD1/MAO-B inhibitor to be used in accordance with the invention is a 2-cyclylcyclopropan-1-amine compound which is a compound of the following formula (VI) or an enantiomer, a diastereomer, or a mixture thereof, or a pharmaceutically acceptable salt or solvate thereof:

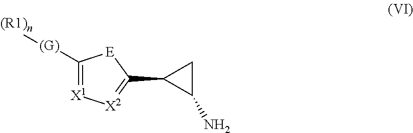

In formula (VI), E is —N(R3)-, —O—, or —S—, or is —X³═X⁴—;
X¹ and X² are independently C(R2) or N;
X³ and X⁴, when present, are independently C(R2) or N;
(G) is a cyclyl group;
each (R1) is independently chosen from alkyl, alkenyl, alkynyl, cyclyl, -L1-cyclyl, L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl; each (R2) is independently chosen from —H, alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl, wherein each (R2) group has 1, 2, or 3 independently chosen optional substituents or two (R2) groups can be taken together to form a heterocyclyl or aryl group having 1, 2, or 3 independently chosen optional substituents, wherein said optional substituents are independently chosen from alkyl, alkanoyl, heteroalkyl, heterocyclyl, haloalkyl, cycloalkyl, carbocyclyl, arylalkoxy, heterocyclylalkoxy, aryl, aryloxy, heterocyclyloxy, alkoxy, haloalkoxy, oxo, acyloxy, carbonyl, carboxyl, carboxamido, cyano, halogen, hydroxyl, amino, aminoalkyl, amidoalkyl, amido, nitro, thiol, alkylthio, arylthio, sulfonamide, sulfinyl, sulfonyl, urea, or carbamate;
R3 is —H or a (C₁-C₆)alkyl group;
each L1 is independently alkylene or heteroalkylene; and
n is 0, 1, 2, 3, 4 or 5.

In a further embodiment, the LSD1 inhibitor or selective LSD1 inhibitor or dual LSD1/MAO-B inhibitor to be used in accordance with the invention is a 2-cyclylcyclopropan-1-amine compound which is a compound of the following formula (VII) or an enantiomer, a diastereomer, or a mixture thereof, or a pharmaceutically acceptable salt or solvate thereof:

(A′)x-(A)-(B)—(Z)-(L)-(D) (VII)

In formula (VII), (A) is heteroaryl or aryl;
each (A′), if present, is independently chosen from aryl, arylalkoxy, arylalkyl, heterocyclyl, aryloxy, halo, alkoxy, haloalkyl, cycloalkyl, haloalkoxy, and cyano, wherein each (A′) is substituted with 0, 1, 2, or 3 substituents independently chosen from halo, haloalkyl, haloalkoxy, aryl, arylalkoxy, alkyl, alkoxy, amido, —CH₂C(═O)NH₂, heteroaryl, cyano, sulfonyl, and sulfinyl;

### X is 0, 1, 2, or 3;

(B) is a cyclopropyl ring, wherein (A) and (Z) are covalently bonded to different carbon atoms of (B);

### (Z) is —NH—;

(L) is chosen from a single bond, —CH₂—, —CH₂CH₂—, —CH₂CH₂CH₂—, and —CH₂CH₂CH₂CH₂—; and
(D) is an aliphatic carbocyclic group or benzocycloalkyl, wherein said aliphatic carbocyclic group or said benzocycloalkyl has 0, 1, 2, or 3 substituents independently chosen from —NH₂, —NH(C₁-C₆ alkyl), —N(C₁-C₆ alkyl)(C₁-C₆ alkyl), alkyl, halo, amido, cyano, alkoxy, haloalkyl, and haloalkoxy;
with the proviso that the following compounds are excluded:
N-(2-phenylcyclopropyl)-cyclopentanamine;
10,11-dihydro-N-(2-phenylcyclopropyl)-5H-dibenzo[a,d]cyclohepten-5-amine; and
trans-N-(2-phenylcyclopropyl)-cyclohexanamine.

In a further embodiment, the LSD1 inhibitor or selective LSD1 inhibitor or dual LSD1/MAO-B inhibitor to be used in accordance with the invention is a 2-cyclylcyclopropan-1-amine compound which is a compound of the following formula (VIII) or a pharmaceutically acceptable salt or solvate thereof:

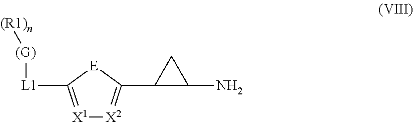

In formula (VII), E is —N(R3)-, —S—, —O—, or —X³═X⁴—;
X¹ and X² are each independently C(R2) or N;
X³ and X¹, when present, are each independently C(R2) or N;

### L1 is —NH— or —NH—CH₂—;

G is a cyclyl group;
each R1 is independently chosen from alkyl, alkenyl, alkynyl, cyclyl, -L2-cyclyl, -L2-amino, -L2-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl; each R2 is independently chosen from —H, alkyl, alkenyl, alkynyl, cyclyl, -L2-cyclyl, -L2-amino, -L2-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl, wherein each R2 group has 1, 2, or 3 independently chosen optional substituents, and further wherein two R2 groups bound to adjacent carbon atoms can be taken together to form a heterocyclyl or aryl group having 1, 2, or 3 independently chosen optional substituents; wherein said optional substituents are each independently chosen from alkyl, alkanoyl, heteroalkyl, heterocyclyl, haloalkyl, cycloalkyl, carbocyclyl, arylalkoxy, heterocyclylalkoxy, aryl, aryloxy, heterocyclyloxy, alkoxy, haloalkoxy, oxo, acyloxy, carbonyl, carboxyl, carboxamido, cyano, halogen, hydroxyl, amino, aminoalkyl, amidoalkyl, amido, nitro, thiol, alkylthio, arylthio, sulfinyl, sulfonyl, sulfonamide, urea or carbamate;
R3 is —H or an (C1-C6)alkyl group;
each L2 is independently chosen from alkylene or heteroalkylene; and
n is 0, 1, 2, 3, 4 or 5.

In a further embodiment, the LSD1 inhibitor or selective LSD1 inhibitor or dual LSD1/MAO-B inhibitor to be used in accordance with the invention is a 2-cyclylcyclopropan-1-amine compound which is a compound of the following formula (IX) or a pharmaceutically acceptable salt or solvate thereof:

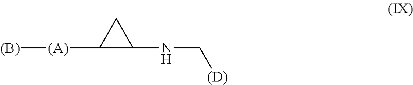

In formula (IX), (A) is a cyclyl group having n substituents (R3);
(B) is a cyclyl group or an -(L1)-cyclyl group, wherein said cyclyl group or the cyclyl moiety comprised in said -(L1)-cyclyl group has n substituents (R2);
(L1) is —O—, —NH—, —N(alkyl)-, alkylene or heteroalkylene;
(D) is a heteroaryl group or an -(L2)-heteroaryl group, wherein said heteroaryl group or the heteroaryl moiety comprised in said -(L2)-heteroaryl group has one substituent (R1), and further wherein said heteroaryl group is covalently bonded to the remainder of the molecule through a ring carbon atom or the heteroaryl moiety comprised in said -(L2)-heteroaryl group is covalently bonded to the (L2) moiety through a ring carbon atom;
(L2) is —O—, —NH—, —N(alkyl)-, alkylene or heteroalkylene;
(R1) is a hydrogen bonding group;
each (R2) is independently selected from alkyl, alkenyl, alkynyl, cyclyl, amino, amido, C-amido, alkylamino, hydroxyl, nitro, halo, haloalkyl, haloalkoxy, cyano, sulfinyl, sulfonyl, sulfonamide, alkoxy, acyl, carboxyl, carbamate or urea;
each (R3) is independently selected from alkyl, alkenyl, alkynyl, cyclyl, amino, amido, C-amido, alkylamino, hydroxyl, nitro, halo, haloalkyl, haloalkoxy, cyano, sulfinyl, sulfonyl, sulfonamide, alkoxy, acyl, carboxyl, carbamate, or urea; and n is independently 0, 1, 2, 3 or 4.

Exemplary non-limiting selective LSD1 inhibitors are OG Compounds A, B, C and D as shown in FIG. 1 as well as pharmaceutically acceptable salts or solvates thereof. Exemplary non-limiting dual LSD1/MAO B selective inhibitors are OG Compounds E, F and G as shown in FIG. 2 as well as pharmaceutically acceptable salts or solvates thereof.

The 2-cyclylcyclopropan-1-amine compounds disclosed and described herein, including, e.g., the compounds of formulae (I) to (IX), can be prepared by methods known in the art of synthetic chemistry. For example, these compounds can be prepared in accordance with or in analogy to the methods described in WO2010/043721, WO2010/084160, WO2011/035941, WO2011/042217, PCT/EP2011/062947, PCT/EP2011/056279, PCT/EP2011/062949, and EP10187039.2.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

As used herein, the term “aryl,” refers a carbocyclic aromatic system containing one ring, or two or three rings fused together where in the ring atoms are all carbon. The term “aryl” groups includes, but is not limited to groups such as phenyl, naphthyl, or anthracenyl.
As used herein, the term “heterocyclyl” or “hetercycle,” each refer to a saturated, partially unsaturated, or fully unsaturated monocyclic, bicyclic, or tricyclic heterocyclic group containing at least one heteroatom as a ring member, wherein each said heteroatom may be independently selected from the group consisting of nitrogen, oxygen, and sulfur wherein the nitron or sulfur atoms may be oxidized (e.g., —N═O, —S(═—O)—, or —S(═O)₂—). Additionally, 1, 2, or 3 of the carbon atoms of the heterocyclyl may be optionally oxidized (e.g., to give an oxo group or ═O). One group of heterocyclyls has from 1 to 4 heteroatoms as ring members. Another group of heterocyclyls has from 1 to 2 heteroatoms as ring members. One group of heterocyclyls has from 3 to 8 ring members in each ring. Yet another group of heterocyclyls has from 3 to 7 ring members in each ring. Again another group of heterocyclyls has from 5 to 6 ring members in each ring. “Heterocyclyl” is intended to encompass a heterocyclyl group fused to a carbocyclyl or benzo ring systems. Examples of heterocyclyl groups include, but are not limited to, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, or imidazolidinyl. Examples of heteroaryls that are heterocyclyls include, but are not limited to, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, or furopyridinyl.
As used herein, the term “heteroaryl,” refers to a 3 to 7 membered unsaturated monocyclic ring, or a fused monocyclic, bicyclic, or tricyclic ring system in which the rings are aromatic and which at least one ring contains at least one atom selected from the group consisting of O, S, and N. One group of heteroaryls has from 5 to 7 carbon atoms. Examples of heteroaryl groups include, but are not limited to, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, or furopyridinyl.
As used herein, the term “acyl,” refers to a carbonyl attached to an alkenyl, alkyl, aryl, cycloalkyl, heteroaryl, heterocyclyl, or any other moiety where the atom attached to the carbonyl is carbon. An “acetyl” group refers to a —C(═O)CH₃ group. An “alkylcarbonyl” or “alkanoyl” group refers to an alkyl group attached to the parent molecular moiety through a carbonyl group. Examples of such groups include, but are not limited to, methylcarbonyl or ethylcarbonyl. Examples of acyl groups include, but are not limited to, formyl, alkanoyl or aroyl.
As used herein, the term “alkenyl,” refers to a straight-chain or branched-chain hydrocarbon group having one or more double bonds and containing from 2 to 20 carbon atoms. A (C2-C6)alkenyl has from 2 to 6 carbon atoms.
As used herein, the term “alkoxy,” refers to an alkyl ether group, wherein the term alkyl is as defined below. Examples of suitable alkyl ether groups include, but are not limited to, methoxy, ethoxy, n-propoxy, isopropoxy, n-butoxy, iso-butoxy, sec-butoxy, tert-butoxy, or n-pentoxy.
As used herein, the term “alkyl,” refers to a straight-chain or branched-chain alkyl group containing from 1 to 20 carbon atoms. A (C1-C10)alkyl has from 1 to 10 carbon atoms and a (C1-C6)alkyl has from 1 to 6 carbon atoms and a (C1-C4)alkyl has from 1 to 4 carbon atoms. Examples of alkyl groups include, but are not limited to, methyl, ethyl, n-propyl, isopropyl, n-butyl, isobutyl, sec-butyl, tert-butyl, pentyl, isopentyl, neo-pentyl, iso-amyl, hexyl, heptyl, octyl, or nonyl.
As used herein, the term “alkylene” refers to an alkyl group attached at two positions, i.e. an alkanediyl group. Examples include, but are not limited to, methylene, ethylene, propylene, butylene, pentylene, hexylene, heptylene, octylene, or nonylene.
As used herein, the term “alkylamino,” refers to an alkyl group attached to the parent molecular moiety through an amino group. Suitable alkylamino groups may be mono- or dialkylated, forming groups including, but not limited to N-methylamino, N-ethylamino, N,N-dimethylamino, N,N-ethylmethylamino, N,N-diethylamino, N-propylamino, and N,N-methylpropylamino.
As used herein, the term “alkynyl,” refers to a straight-chain or branched-chain hydrocarbon group having one or more triple bonds and containing from 2 to 20 carbon atoms. A (C2-C6)alkynyl has from 2 to 6 carbon atoms. A (C2-C4)alkynyl has from 2 to 4 carbon atoms. Examples of alkynyl groups include, but are not limited to, ethynyl, propynyl, hydroxypropynyl, butyn-1-yl, butyn-2-yl, pentyn-1-yl, 3-methylbutyn-1-yl, or hexyn-2-yl.
As used herein, the terms “amido” and “carbamoyl,” refer to an amino group as described below attached to the parent molecular moiety through a carbonyl group (e.g., —C(═O)NRR′), or vice versa (—N(R)C(═O)NR′). “Amido” and “carbamoyl” encompass “C-amido”, “N-amido” and “acylamino” as defined herein. R and R′ are as defined herein.
As used herein, the term “C-amido,” refers to a —C(═O)NRR′ group with R and R′ as defined herein.
As used herein, the term “amino,” refers to —NRR′, wherein R and R′ are independently selected from the group consisting of hydrogen, alkyl, heteroalkyl, aryl, carbocyclyl, and heterocyclyl. Additionally, R and R′ may be combined to form a heterocyclyl.
As used herein, the term “arylalkoxy” or “aralkoxy,” refers to an aryl group attached to the parent molecular moiety through an alkoxy group. Examples of arylalkoxy groups include, but are not limited to, benzyloxy or phenethoxy.
As used herein, the term “arylalkyl” or “aralkyl,” refers to an aryl group attached to the parent molecular moiety through an alkyl group.
As used herein, the term “aryloxy,” refers to an aryl group attached to the parent molecular moiety through an oxy (—O—).
As used herein, the term “carbamate,” refers to an O-carbamyl or N-carbamyl group as defined herein.
As used herein, the term “carbonyl,” when alone includes formyl —C(═O)H and in combination is a —C(═O)— group.
As used herein, the term “carboxyl” or “carboxy” refers to —C(═O)OH or the corresponding “carboxylate” anion, such as is in a carboxylic acid salt. An “O-carboxy” group refers to a RC(═O)O— group, where R is as defined herein. A “C-carboxy” group refers to a —C(═O)OR groups where R is as defined herein.
As used herein, the term “cyano” refers to —CN.
As used herein, the term “carbocyclyl” refers to a saturated or partially saturated monocyclic or a fused bicyclic or tricyclic group wherein the ring atoms of the cyclic system are all carbon and wherein each cyclic moiety contains from 3 to 12 carbon atom ring members. “Carbocyclyl” encompasses benzo fused to a carbocyclyl ring system. One group of carbocyclyls have from 5 to 7 carbon atoms. Examples of carbocyclyl groups include, but are not limited to, cyclopropyl, cyclobutyl, cyclopentyl, cyclohexyl, and cycloheptyl, tetrahydronapthyl, indanyl, octahydronaphthyl, 2,3-dihydro-1H-indenyl, or adamantyl.
As used herein, the term “cycloalkyl” refers to a saturated monocyclic, bicyclic or tricyclic group wherein the ring atoms of the cyclic system are all carbon and wherein each cyclic moiety contains from 3 to 12 carbon atom ring members. One group of cycloalkyls has from 5 to 7 carbon atoms. Examples of cycloalkyl groups include, but are not limited to, cyclopropyl, cyclobutyl, cyclopentyl, cyclohexyl, cycloheptyl, or adamantyl.
As used herein, the term “cycloalkenyl” refers to a partially saturated monocyclic, bicyclic or tricyclic group wherein the ring atoms of the cyclic system are all carbon and wherein each cyclic moiety contains from 3 to 12 carbon atom ring members. One group of carboalkenyls have from 5 to 7 carbon atoms. Examples of cycloalkenyl groups include, but are not limited to, cyclobutenyl, cyclopentenyl, or cyclohexenyl.
As used herein, the term “cyclyl” refers to an aryl, heterocyclyl, or carbocyclyl group as defined herein. A “cyclyl” group may, for example, be an aryl group, a cycloalkyl group, a heteroaryl group or a heterocycloalkyl group.
As used herein, the term “halo” or “halogen” refers to fluorine, chlorine, bromine, or iodine.
As used herein, the term “haloalkoxy” refers to a haloalkyl group attached to the parent molecular moiety through an oxygen atom. Examples of haloalkoxy groups include, but are not limited to, trifluoromethoxy, 2-fluoroethoxy, or 3-chloropropoxy.
As used herein, the term “haloalkyl” refers to an alkyl group having the meaning as defined above wherein one or more hydrogens are replaced with a halogen. Specifically embraced are monohaloalkyl, dihaloalkyl or polyhaloalkyl groups. A monohaloalkyl group, for one example, may have an iodo, bromo, chloro or fluoro atom within the group. Dihalo or polyhaloalkyl groups may have two or more of the same halo atoms or a combination of different halo groups. Examples of haloalkyl groups include, but are not limited to, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl or dichloropropyl.
As used herein, the term “heteroalkyl” refers to a straight or branched alkyl chain, wherein one, two, or three carbons forming the alkyl chain are each replaced by a heteroatom independently selected from the group consisting of O, N, and S, and wherein the nitrogen and/or sulfur heteroatom(s) (if present) may optionally be oxidized and the nitrogen heteroatom(s) (if present) may optionally be quaternized. The heteroatom(s) O, N and S may, for example, be placed at an interior position of the heteroalkyl group, i.e., the heteroalkyl may be bound to the remainder of the molecule via a carbon atom. Up to two heteroatoms may be consecutive, such as, for example, —CH₂—NH—OCH₃.
As used herein, the term “heteroalkylene” refers to a heteroalkyl group attached at two positions. Examples include, but are not limited to, —CH₂OCH₂—, —CH₂SCH₂—, and —CH₂NHCH₂—, —CH₂S—, or —CH₂—NHCH(CH₃)CH₂—.
As used herein, the term “heterocycloalkyl,” refers to a heterocyclyl group that is not fully saturated e.g., one or more of the rings systems of a heterocycloalkyl is not aromatic. Examples of heterocycloalkyls include piperazinyl, morpholinyl, piperidinyl, or pyrrolidinyl.
As used herein, the term “hydroxyl,” as used herein, refers to —OH.
As used herein, the term “hydroxyalkyl,” as used herein, refers to a hydroxyl group attached to the parent molecular moiety through an alkyl group.
As used herein, the phrase “in the main chain,” refers to the longest contiguous or adjacent chain of carbon atoms starting at the point of attachment of a group to the compounds of any one of the formulas disclosed herein.
As used herein, the term phrase “linear chain of atoms” refers to the longest straight chain of atoms independently selected from carbon, nitrogen, oxygen and sulfur.
As used herein, the term “lower,” where not otherwise specifically defined, means containing from 1 to and including 6 carbon atoms.
As used herein, the term “lower aryl,” means phenyl or naphthyl.
As used herein, the term “lower heteroaryl,” means either 1) monocyclic heteroaryl comprising five or six ring members, of which between one and four said members may be heteroatoms selected from O, S, or N.
As used herein, the terms “benzo” and “benz,” refer to the divalent group C₆H₄═ derived from benzene. Examples include, but are not limited to, benzothiophene or benzimidazole.
As used herein, the term “nitro,” refers to —NO₂.
As used herein, the terms “sulfonate” “sulfonic acid” and “sulfonic,” refers to the —SO₃H group and its anion as the sulfonic acid is used in salt formation.
As used herein, the term “sulfanyl,” to —S—.
As used herein, the term “sulfinyl,” refers to —S(═O)(R)—, with R as defined herein.
As used herein, the term “sulfonyl,” refers to —S(═O)₂R, with R as defined herein,
As used herein, the term “sulfonamide”, refers to an N-sulfonamido or S-sulfonamido group as defined herein.
S As used herein, the term “urea,” refers to a —N(R)C(═O)N(R) group wherein R and R′ are as defined herein.
As used herein, “hydrogen bonding group” refers to a substituent group, which is capable of taking part in a non-covalent bonding between hydrogen and another atom (usually nitrogen or oxygen). Examples include, but are not limited to, —OH, NH₂, —OH, amido, —S(O)₂NH₂, —C(═O)NH₂, —CH₂—C(═O)NH₂, and —CH₂—NH₂.
As used herein, the term “optionally substituted” means the preceding or anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxyl, amino, lower alkylamino, arylamino, aminoalkyl, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, carbamate, and urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.” In one specific definition, the optional substituents are chosen from hydroxyl, halo, alkyl, alkoxy, haloalkyl, haloalkoxy, —N((C1-C3)alkyl)₂, —NH((C1-C3)alkyl), —NHC(═O)((C1-C3)alkyl), —C(═O)OH, —C(═O)O((C1-C3)alkyl), —C(═O)(C1-C3)alkyl), —C(═O)NH₂, —C(═O)NH(C1-C3)alkyl), —C(═O)NH(cycloalkyl), —C(═O)N(C1-C3)alkyl)₂, —S(═O)₂((C1-C3)alkyl), —S(═O)₂NH₂, —S(═O)₂N((C1-C3)alkyl)₂, —S(═O)₂NH((C1-C3)alkyl), —CHF₂, —OCF₃, —OCHF₂, —SCF₃, —CF₃, —CN, —NH₂, —NO₂, or tetrazolyl.
The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl. Whether an R group has a number designation or not, every R group, including R, R′ and R^{p} where p=(1, 2, 3, . . . p), every substituent, and every term should be understood to be independent of every other in terms of selection from a group.
Should any variable, substituent, or term (e.g., aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(═O)N(R)— may be attached to the parent moiety at either the carbon or the nitrogen.
As used herein, the term “2-cyclylcyclopropan-1-amine compound” refers to a compound comprising a 2-cyclylcyclopropan-1-amine moiety or a pharmaceutically acceptable salt or solvate thereof. Exemplary 2-cyclylcyclopropan-1-amine compounds are, without limitation, 2-arylcyclopropan-1-amine compounds (such as 2-phenylcyclopropan-1-amine compounds) and 2-heteroarylcyclopropan-1-amine compounds (such as 2-pyridinylcyclopropan-1-amine compounds).
As used herein, the term “2-arylcyclopropan-1-amine compound” refers to a compound comprising a 2-arylcyclopropan-1-amine moiety or a pharmaceutically acceptable salt or solvate thereof.
As used herein, the term “2-heteroarylcyclopropan-1-amine compound” refers to a compound comprising a 2-heteroarylcyclopropan-1-amine moiety or a pharmaceutically acceptable salt or solvate thereof.
As used herein, the term “2-phenylcyclopropan-1-amine compound” refers to a compound comprising a 2-phenylcyclopropan-1-amine moiety or a pharmaceutically acceptable salt or solvate thereof.
As used herein, the term “2-pyridinylcyclopropan-1-amine compound” refers to a compound comprising a 2-pyridinylcyclopropan-1-amine moiety or a pharmaceutically acceptable salt or solvate thereof.
As used herein, the term “phenelzine compound” refers to a compound comprising a 2-phenylethylhydrazine moiety or a pharmaceutically acceptable salt or solvate thereof.
As used herein, the term “propargylamine compound” refers to a compound comprising a propargylamine moiety or a pharmaceutically acceptable salt or solvate thereof. An exemplary propargylamine compound is, without limitation, pargyline (N-benzyl-N-methylprop-2-yn-1-amine).
As used herein, the term “LSD1 selective inhibitor” or “selective inhibitor of LSD1” refers to an LSD1 inhibitor which preferably has an IC50 value for LSD1 that is at least two-fold lower than its IC50 values for MAO-A and MAO-B. More preferably, an LSD1 selective inhibitor has an IC50 value for LSD1 which is at least five-fold lower than its IC50 values for MAO-A and MAO-B. Even more preferably, an LSD1 selective inhibitor has an IC50 value for LSD1 which is at least ten-fold lower than its IC50 values for MAO-A and MAO-B. Even more preferably, an LSD1 selective inhibitor has an IC50 value for LSD1 which is at least 20-fold lower than its IC50 values for MAO-A and MAO-B. Even more preferably, an LSD1 selective inhibitor has an IC50 value for LSD1 which is at least 50-fold lower than its IC50 values for MAO-A and MAO-B. Even more preferably, an LSD1 selective inhibitor has an IC50 value for LSD1 which is at least 100-fold lower than its IC50 values for MAO-A and MAO-B. The ability of a compound to inhibit LSD1 and its IC50 values for LSD1, MAO-A and MAO-B are preferably to be determined in accordance with the experimental protocol described in Example 1.
As used herein, the term “dual LSD1/MAO-B selective inhibitor” or “dual LSD1/MAO-B inhibitor” or “dual inhibitor selective for LSD1 and MAO-B” or “dual inhibitor of LSD1 and MAO-B” refers to an LSD1 inhibitor which preferably has IC50 values for LSD1 and MAO-B which are at least two-fold lower than its IC50 value for MAO-A. More preferably, a dual LSD1/MAO-B selective inhibitor has IC50 values for LSD1 and MAO-B which are at least five-fold lower than its IC50 value for MAO-A. Even more preferably, a dual LSD1/MAO-B selective inhibitor has IC50 values for LSD1 and MAO-B which are at least ten-fold lower than its IC50 value for MAO-A. Even more preferably, a dual LSD1/MAO-B selective inhibitor has IC50 values for LSD1 and MAO-B which are at least 20-fold lower than its IC50 value for MAO-A. The ability of a compound to inhibit LSD1 and MAO-B and its IC50 values for LSD11, MAO-A and MAO-B are preferably to be determined in accordance with the experimental protocol described in Example 1.

The selective LSD1 and dual LSD1/MAO-B inhibitors for use in the invention desirably inhibit LSD1 and/or MAO-B selectively compared to MAO-A, thus avoiding deleterious side effects associated with administration to animals, including humans, of MAO-A inhibitors. As the inventors have described herein, the selective LSD1 inhibitors and the dual LSD1/MAO-B inhibitors can be administered in a such a way to an individual, e.g., a mammal or human, to achieve concentration in vivo that are expected to inhibit LSD1 and/or MAO-B while avoiding the toxicity associated with inhibition of MAO-A and these concentrations are sufficient enough to improve specific phenotypes or symptoms associated with protein conformation disorders.

The invention provides a pharmaceutical composition comprising a pharmaceutically acceptable carrier and a compound which is a selective inhibitor of LSD1. Preferably, LSD1 selective inhibitors have IC50 values for LSD1 which are at least two-fold lower than the IC50 value for MAO-A and/or MAO-B. Even more preferably, LSD1 selective inhibitors have IC50 values for LSD1 which are at least five-fold lower than the IC50 value for MAO-A and/or MAO-B. Yet even more preferably, LSD1 selective inhibitors have IC50 values for LSD1 which are at least ten-fold lower than the IC50 value for MAO-A and/or MAO-B. The ability of a compound to inhibit LSD1 and its IC50 values for LSD1, MAO-A and MAO-B can be determined in accordance with the experimental protocol described in Example 1.

The invention also provides a pharmaceutical composition comprising a pharmaceutically acceptable carrier and a compound which is a dual inhibitor selective for LSD1 and MAO-B. Preferably, dual LSD1/MAO-B selective inhibitors have IC50 values for LSD1 and MAO-B which are at least two-fold lower than the IC50 value for MAO-A. Even more preferably, dual LSD1/MAO-B selective inhibitors (i.e., dual LSD1/MAO-B inhibitors) have IC50 values for LSD1 and MAO-B which are at least five-fold lower than the IC50 value for MAO-A. Yet even more preferably, dual LSD1/MAO-B selective inhibitors have IC50 values for LSD1 and MAO-B which are at least ten-fold lower than the IC50 value for MAO-A. The ability of a compound to inhibit LSD1 and MAO-B and its IC50 values for LSD1, MAO-A and MAO-B can be determined in accordance with the experimental protocol described in Example 1.

Typically, compounds for use as selective LSD1 inhibitors or dual inhibitors of LSD1 and MAO-B can be effective at an amount of from about 0.01 μg/kg to about 100 mg/kg per day based on total body weight. The active ingredient may be administered at once, or may be divided into a number of smaller doses to be administered at predetermined intervals of time. The suitable dosage unit for humans for each administration can be, e.g., from about 1 μg to about 2000 mg, preferably from about 5 μg to about 1000 mg, and even more preferably from about 0.5 mg to about 500 mg. The active ingredient can be administered orally or by other routes of administration e.g., IP, IV, etc. Preferably, the inhibitor is formulated and delivered in such a way as to achieve concentration in vive that modulate the target activity, e.g., LSD1 and/or MAO-B. Thus, in a specific embodiment, the effective amount of compound ranges from 0.05 μg/kg to about 100 mg/kg, preferably from 0.05 μg/kg to about 50 mg/kg.

It should be understood that the dosage ranges set forth above are exemplary only and are not intended to limit the scope of this invention unless specified. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can be adjusted as the various factors change over time.

For oral delivery, the active compounds can be incorporated into a formulation that includes pharmaceutically acceptable carriers such as binders (e.g., gelatin, cellulose, gum tragacanth), excipients (e.g., starch, lactose), lubricants (e.g., magnesium stearate, silicon dioxide), disintegrating agents (e.g., alginate, Primogel, and corn starch), and sweetening or flavoring agents (e.g., glucose, sucrose, saccharin, methyl salicylate, and peppermint). The formulation can be orally delivered in the form of enclosed gelatin capsules or compressed tablets. Capsules and tablets can be prepared in any conventional techniques. The capsules and tablets can also be coated with various coatings known in the art to modify the flavors, tastes, colors, and shapes of the capsules and tablets. In addition, liquid carriers such as fatty oil can also be included in capsules.

Suitable oral formulations can also be in the form of suspension, syrup, chewing gum, wafer, elixir, and the like. If desired, conventional agents for modifying flavors, tastes, colors, and shapes of the special forms can also be included. In addition, for convenient administration by enteral feeding tube in patients unable to swallow, the active compounds can be dissolved in an acceptable lipophilic vegetable oil vehicle such as olive oil, corn oil and safflower oil.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacteria agents, surfactants, and antioxidants can all be included. For example, useful components include sodium chloride, acetates, citrates or phosphates buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampoules.

Routes of topical administration include nasal, buccal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like. A special form of topical administration is delivery by a transdermal patch. Methods for preparing transdermal patches are disclosed, e.g., in Brown et al. (1988), Ann. Rev. Med. 39:221-229, which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al. (1984), J. Clin. Psych. 45:242-247. Hydrogels can be used as a carrier for the sustained release of the active compounds. Hydrogels are generally known in the art. They are typically made by cross-linking high molecular weight biocompatible polymers into a network, which swells in water to form a gel-like material. Preferably, hydrogels are biodegradable or biosorbable. For purposes of this invention, hydrogels made of polyethylene glycols, collagen, or poly(glycolic-co-L-lactic acid) may be useful. See, e.g., Phillips et al. (1984), J. Pharmaceut. Sci. 73:1718-1720.

The active compounds can also be conjugated, to a water soluble non-immunogenic non-peptidic high molecular weight polymer to form a polymer conjugate. For example, an active compound is covalently linked to polyethylene glycol to form a conjugate. Typically, such a conjugate exhibits improved solubility, stability, and reduced toxicity and immunogenicity. Thus, when administered to a patient, the active compound in the conjugate can have a longer half-life in the body, and exhibit better efficacy. See generally, Burnham (1994), Am. J. Hosp. Pharm. 15:210-218. PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). It is preferred that the covalent linkage between the polymer and the active compound and/or the polymer itself is hydrolytically degradable under physiological conditions. Such conjugates known as “prodrugs” can readily release the active compound inside the body. Controlled release of an active compound can also be achieved by incorporating the active ingredient into microcapsules, nanocapsules, or hydrogels generally known in the art. Other pharmaceutically acceptable prodrugs of the compounds of this invention include, but are not limited to, esters, carbonates, thiocarbonates, N-acyl derivatives, N-acyloxyalkyl derivatives, quaternary derivatives of tertiary amines, N-Mannich bases, Schiff bases, aminoacid conjugates, phosphate esters, metal salts and sulfonate esters.

Liposomes can also be used as carriers for the active compounds of the present invention. Liposomes are micelles made of various lipids such as cholesterol, phospholipids, fatty acids, and derivatives thereof. Various modified lipids can also be used. Liposomes can reduce the toxicity of the active compounds, and increase their stability. Methods for preparing liposomal suspensions containing active ingredients therein are generally known in the art. See, e.g., U.S. Pat. No. 4,522,811; Prescott, Ed., Methods in Cell Biology, Volume XIV, Academic Press, New York, N. Y. (1976).

The active ingredient can be formulated as a pharmaceutically acceptable salt. A “pharmaceutically acceptable salt” is intended to mean a salt that retains the biological effectiveness of the free acids and bases of the specified compound and that is not biologically or otherwise undesirable. A compound for use in the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrophosphates, dihydrophosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4 dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, gamma-hydroxybutyrates, glycollates, tartrates, methane-sulfonates, propanesulfonates, naphthalene-1-sulfonates, naphthalene-2-sulfonates, or mandelates.

As used herein, a “pharmaceutically acceptable carrier” refers to a non-API (API refers to Active Pharmaceutical Ingredient) substances such as disintegrators, binders, fillers, and lubricants used in formulating pharmaceutical products. They are generally safe for administering to humans according to established governmental standards, including those promulgated by the United States Food and Drug Administration and the European Medical Agency.

The active compounds can also be administered in combination with another active agent that synergistically treats or prevents the same symptoms or is effective for another disease or symptom in the patient treated so long as the other active agent does not interfere with or adversely affect the effects of the active compounds of this invention. Such other active agents include but are not limited to anti-inflammation agents, antiviral agents, antibiotics, antifungal agents, antithrombotic agents, cardiovascular drugs, cholesterol lowering agents, anti-cancer drugs, hypertension drugs, and the like.

Preferably, the compounds for use in the methods of the invention have molecular weights of less than 700 daltons and more preferably less than 500 daltons.

There examples described herein are intended to illustrate different aspects of the invention by exemplification and are not intended to limit the scope of the claims or invention.

### EXAMPLES

### Example 1

### Biochemical Assays

Compounds for use in the methods of the invention can be identified by their ability to inhibit LSD1 and/or MAO-B selectively as compared to MAO-A. The ability of the compounds of the invention to inhibit LSD1 can be tested as follows. Human recombinant LSD1 protein was purchased from BPS Bioscience Inc. In order to monitor LSD1 enzymatic activity and/or its inhibition rate by our inhibitor(s) of interest, di-methylated H3-K4 peptide (Millipore) was chosen as a substrate. The demethylase activity was estimated, under aerobic conditions, by measuring the release of H₂O₂ produced during the catalytic process, using the Amplex® Red peroxide/peroxidase-coupled assay kit (Invitrogen). Briefly, a fixed amount of LSD1 was incubated on ice for 15 minutes, in the absence and/or in the presence of various concentrations of inhibitor (e.g., from 0 to 75 M, depending on the inhibitor strength). Tranylcypromine (Biomol International) was used as a control for inhibition. Within the experiment, each concentration of inhibitor was tested in triplicate.

After leaving the enzyme interacting with the inhibitor, 12.5 μM of di-methylated H3-K4 peptide was added to each reaction and the experiment was left for one hour at 37° C. in the dark. The enzymatic reactions were set up in a 50 mM sodium phosphate, pH 7.4 buffer. At the end of the incubation, Amplex® Red reagent and horseradish peroxidase (HPR) solution were added to the reaction according to the recommendations provided by the supplier (Invitrogen), and left to incubate for 30 extra minutes at room temperature in the dark. A 1 μM H₂O₂ solution was used as a control of the kit efficiency. The conversion of the Amplex® Red reagent to resorufin due to the presence of H₂O₂ in the assay, was monitored by fluorescence (excitation at 540 nm, emission at 590 nm) using a microplate reader (Infinite 200, Tecan). Arbitrary units were used to measure level of H₂O₂ produced in the absence and/or in the presence of inhibitor. The maximum demethylase activity of LSD1 was obtained in the absence of inhibitor and corrected for background fluorescence in the absence of LSD1. The Ki (IC50) of each inhibitor was estimated at half of the maximum activity.

Human recombinant monoamine oxidase proteins MAO-A and MAO-B were purchased from Sigma Aldrich. MAOs catalyze the oxidative deamination of primary, secondary and tertiary amines. In order to monitor MAO enzymatic activities and/or their inhibition rate by inhibitor(s) of interest, a fluorescent-based (inhibitor)-screening assay was set up. 3-(2-Aminophenyl)-3-oxopropanamine (kynuramine dihydrobromide, Sigma Aldrich), a non fluorescent compound was chosen as a substrate. Kynuramine is a non-specific substrate for both MAOs activities. While undergoing oxidative deamination by MAO activities, kynuramine is converted into 4-hydroxyquinoline (4-HQ), a resulting fluorescent product.

The monoamine oxidase activity was estimated by measuring the conversion of kynuramine into 4-hydroxyquinoline. Assays were conducted in 96-well black plates with clear bottom (Corning) in a final volume of 100 μL. The assay buffer was 100 mM HEPES, pH 7.5. Each experiment was performed in triplicate within the same experiment. Briefly, a fixed amount of MAO (0.25 μg for MAO-A and 0.5 μg for MAO-B) was incubated on ice for 15 minutes in the reaction buffer, in the absence and/or in the presence of various concentrations of inhibitor (e.g., from 0 to 50 μM, depending on the inhibitor strength). Tranylcypromine (Biomol International) was used as a control for inhibition. After leaving the enzyme(s) interacting with the inhibitor, 60 to 90 μM of kynuramine was added to each reaction for MAO-B and MAO-A assay respectively, and the reaction was left for one hour at 37° C. in the dark. The oxidative deamination of the substrate was stopped by adding 50 μL (v/v) of NaOH 2N. The conversion of kynuramine to 4-hydroxyquinoline, was monitored by fluorescence (excitation at 320 nm, emission at 360 nm) using a microplate reader (Infinite 200, Tecan). Arbitrary units were used to measure levels of fluorescence produced in the absence and/or in the presence of inhibitor. The maximum of oxidative deamination activity was obtained by measuring the amount of 4-hydroxyquinoline formed from kynuramine deamination in the absence of inhibitor and corrected for background fluorescence in the absence of MAO enzymes. The Ki (TC50) of each inhibitor was determined at Vmax/2.

### Example 2

### LSD1 and LSD11/MAO-B Dual Inhibitors

| Compound | LSD1 | MAO-A | MAO-B |
|---|---|---|---|
| No | IC50 (uM) | IC50 (uM) | IC50 (uM) |
| Dual-1 | <0.20 | >1.0 | <0.20 |
| Dual-2 | <0.20 | >40 | <0.30 |
| Selective-1 | <0.10 | >1.0 | >1.0 |
| Selective-2 | <0.10 | >1.0 | >1.0 |

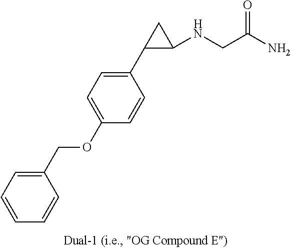 [SMILES: NC(=O)CNC1CC1c1ccc(OCc2ccccc2)cc1]

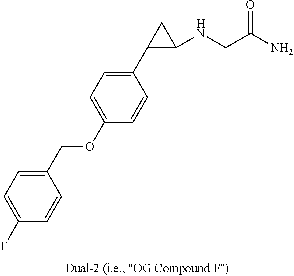 [SMILES: NC(=O)CNC1CC1c1ccc(OCc2ccc(F)cc2)cc1]

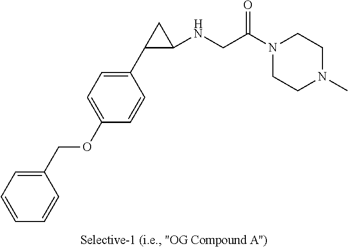 [SMILES: CN1CCN(C(=O)CNC2CC2c2ccc(OCc3ccccc3)cc2)CC1]

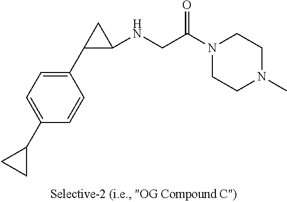 [SMILES: CN1CCN(C(=O)CNC2CC2c2ccc(C3CC3)cc2)CC1]

### Example 3

### LSD1 and LSD1/MAO-B Dual Inhibitors Increase Levels of Dimethylated Histone Lysine in Cell Based Assays

Histone from SH-SY5Y cells grown in the presence of Compound Dual-1 (a dual LSD1/MAO-B inhibitor) or tranylcypromine (parnate) for one, two, and three days were extracted and subjected to western blot analysis using a commercially available antibody specific for dimethylated H-K4. B-actin was used as a loading control.

The results of a western blot stained for H3K4 methylation with SH-SY5Y cells grown in the presence of Compound Dual-1 or tranylcypromine (parnate) for one, two, and three days show that this compound, Dual-1, increases H3K4 methylation in cells in a time dependent manner. Furthermore, Compound Dual-1 appears to be ten-fold or more potent at increasing global dimethylated H3K4 levels as compared to tranylcypromine.

Furthermore, the inventors have conducted similar studies for other dual inhibitors of LSD1/MAO-B and with selective LSD1 inhibitors and found that these compounds can increase dimethylated H3K4 levels in similarly performed assays.

### Example 4

### LSD1 and LSD1/MAO-B Dual Inhibitors Ameliorate Eye Degeneration in Huntington Disease Fly Lines

The eye of the fruit fly, Drosophila melanogaster, provides an ideal model system for studying neuronal survival. Each eye consists of approximately 800 units called ommatidia arranged in a regular pattern and the photoreceptors that compose each ommatidium exist in an ordered trapezoidal array. The presence and organization of these cells can be readily assessed using a technique called optical neutralization of the cornea (Franceschini and Kirschfeld, 1971).

This technique is being used in a screen for compounds that can rescue the neurodegenerative phenotype of a Drosophila model of Huntingtons disease (HD), fly line B-8533 (Jackson et al., 1998). This line contains a P-element construct inserted on the second chromosome, P{w+ gmr.HD-Q120)}. The construct contains a w+ visible eye-color marker, which enables confirmation of the presence of the insertion. This construct also contains the first 171 codons of the human huntingtin (htt)gene (exons 2, 3, and a portion of 4) followed by 120 CAG repeats directly fused to a GMR (glass multimer reporter) enhancer (HDQ120, ibid.). GMR drives expression in the developing larval eye disk in all cells behind the morphogenetic furrow and also in the adult eye (Ellis et al., 1993). Ectopic expression of HD-Q120 in the Drosophila eye disk and eye results in progressive degeneration of the rhabdomeres. HD-Q20 lines display normal external eye and retinal morphology at eclosion; however progressive degeneration of photoreceptor neurons begins at day 2. Histology at ten days reveals disruption of retinal morphology, degenerating photoreceptor cell bodies, and loss of rhabdomeres. Ultrastructural analysis of the degenerating photoreceptors reveals nuclear and cytoplasmic condensation and chromatin clumping characteristic of apoptotic cells.

Specifically, the screen is accomplished as follows: Newly enclosed B-8533 flies are collected daily for five to seven days and kept well fed at 19° C. On day 10, fly food is prepared: (9.3 g agar, 32.4 g sucrose, 61.2 g cornmeal, 129.4 g dextrose) 23.2 g of the fly food mix is used per 100 ml distilled water, this is microwaved two minutes on maximum with frequent stirring, then two minutes on minimum. The food is placed in a 65° water bath, once cooled, 1 ml 10% methyl paraben in EtOH is added and mixed. Twenty-five ml are transferred to a 50 ml centrifuge tube, 25 μl 1000× compound (at two different concentrations) added and mixed (food coloring is added at this time to be sure of sufficient mixing). Five ml of food containing compound is poured into each fly vial for four replicate vials per trial. Once the food has solidified, yeast is added to the top for feeding parent flies.

The now three- to ten-day-old flies are added to the vials (six to ten females and three males in each), incubated at 25° for seven to ten days. When the first pupae begin to darken, the parents are removed from the vials. F1 eclosion-start date is noted and males and females are counted. F1 virgin females are collected daily and placed in vials containing 2 ml food containing test compound or control. Animals are put on fresh food every two days. Day 2 and day 7 animals are examined for rescue of photoreceptor degeneration phenotype by optic neutralization: Heads of flies are truncated and attached with clear nail polish to glass microscope slide to expose the frontal surface. A condensed beam of light is focused through the back of the eye, and is examined at high magnification using a bright-field microscope. Typically 25 to 40 ommatidia per eye can be sampled because of the angle between ommatidia and consequent curvature of the eye. Ommatidia are scored for number of visible rhabdomeres (0 to 7). A minimum of 100 ommatidia from at least six flies is required (preferably 250 to 400 ommatidia from 10 to 20 flies). Significance can be determined by the Mann-Whitney U-test or one-tailed student's T-test. Unused flies are dissected and fixed for ultra-thin sections of the head.

Once the primary screen has been completed, interesting candidates can be tested for toxicity and rescue of additional aspects of HD (motor ability, longevity) can be analyzed in flies using the UAS-Gal4 binary expression system (A. H. Brand and N. Perrimon 1993), wherein truncated Htt with various Q-repeat lengths and pure polyglutamine peptides can be expressed in additional specific tissues and developmental stages (i.e., panneuronal, CNS, motor neurons, muscle cells, etc.).

For more information regarding this type of model system see, e.g., N. Franceschini and K. Kirschfeld (1971), in vivo optical study of photoreceptor elements in the compound eye of Drosophila, Kybernetic 8:1-13; G. R. Jackson et al. (1998), Polyglutamine-expanded human Huntingtin transgenes induce degeneration of Drosophila photoreceptor neurons, Neuron 21:633-642; M. C. Ellis, E. M. O'Neill, and G. M. Rubin (1993), Expression of Drosophila Glass protein and evidence for negative regulation of its activity in non-neuronal cells by another DNA-binding protein, Development 119:855-865; and A. H. Brand and N. Perrimon (1993), Targeted gene expression as a means of altering cell fates and generating dominant phenotypes, Development 118:401-415.

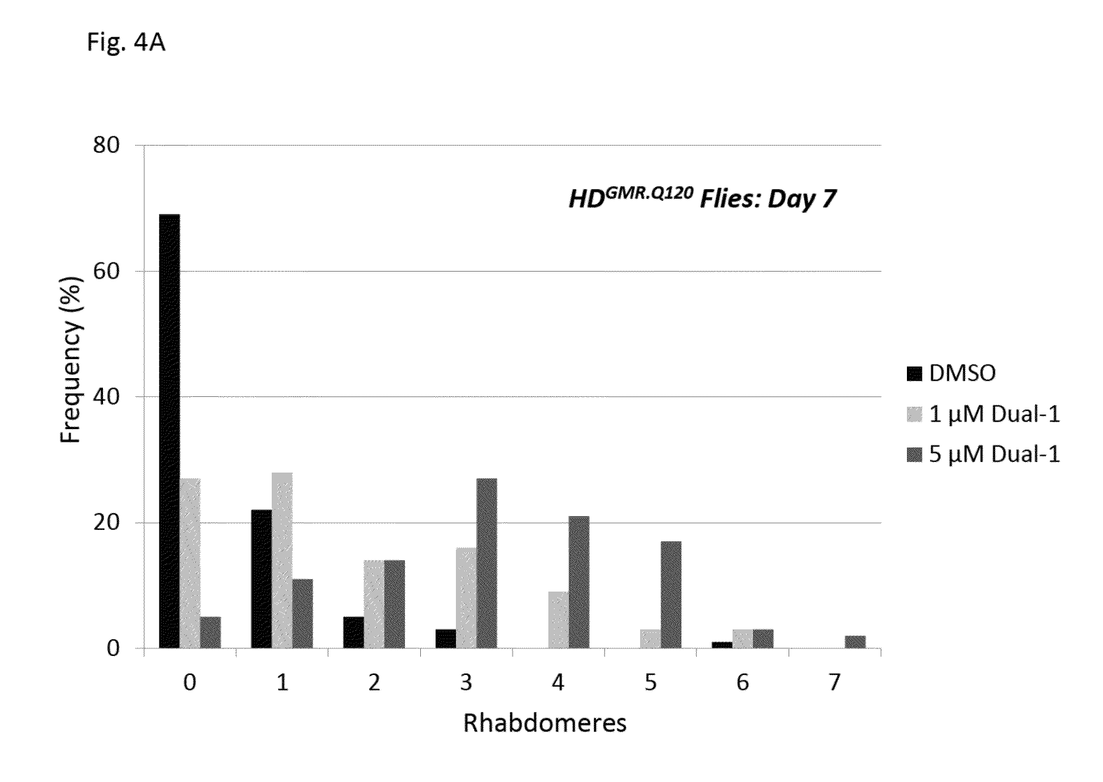

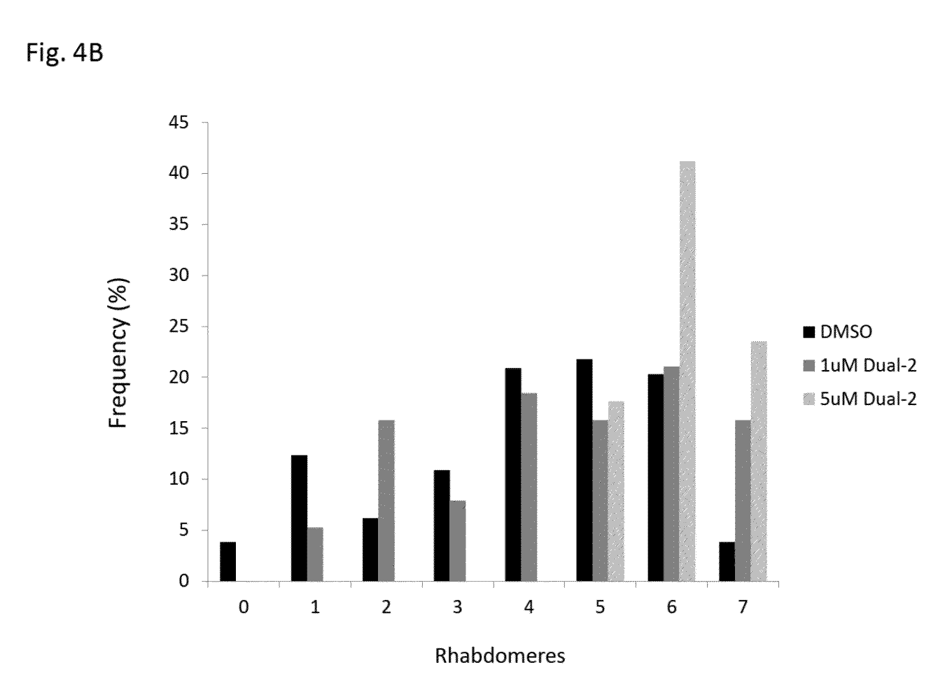

Results from these experiments are shown in FIG. 4. As can be seen, several chemically distinct dual inhibitors of LSD1 and MAO-B improve the eye degeneration phenotype seen in these flies expressing a huntingtin gene expected to have aberrant protein conformation, specifically in the eye. More specifically, the results shown in FIG. 4A that Compound Dual-1 in a concentration dependent manner rescues the eye degeneration phenotype as compared to vehicle treated fly. The results shown in FIG. 4B show that Compound Dual-2 in a concentration dependent manner rescues the eye degeneration phenotype as compared to vehicle treated fly. Wild-type flies have all of their rhabdomeres (7) at high frequency (close to 100%)

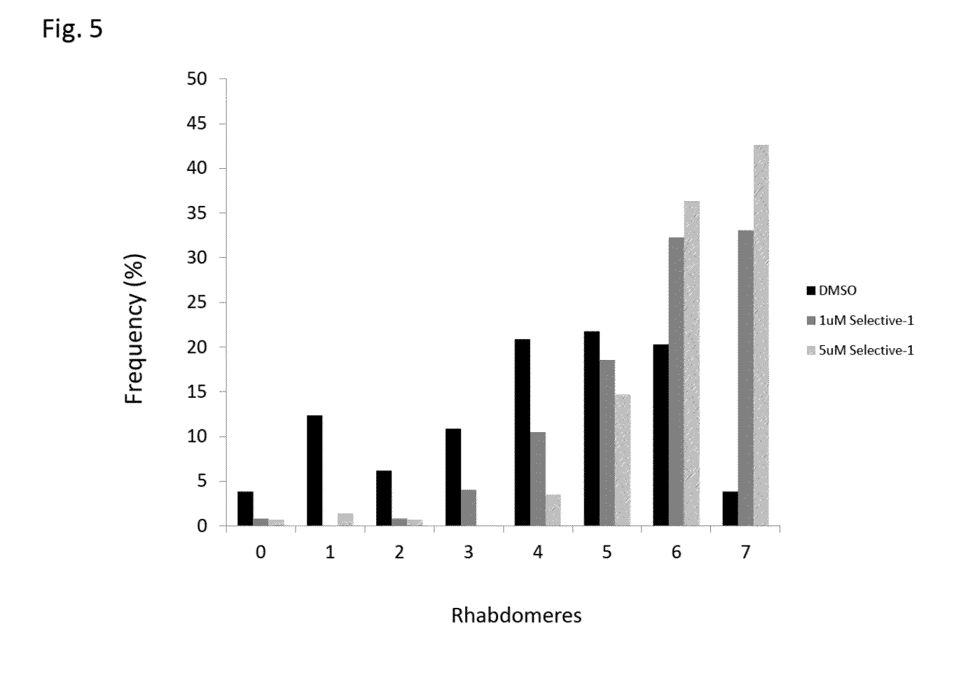

Furthermore, the results shown in FIG. 5 that Compound Selective-1 (a selective LSD1 inhibitor) in a concentration dependent manner rescues the eye degeneration phenotype as compared to vehicle treated fly.

Thus, in sum, these fly results show that inhibitors designed to selectively inhibit LSD1 or LSD1 and MAO-B rescue a biochemical “defect” caused by or associated with a protein conformational disorder.

Generally speaking, rescue effects are seen at day 2 and also at day 7 for the selective LSD1 inhibitors and the dual LSD1/MAO-B inhibitors.

### Example 5

### LSD1 Inhibitors Lessen Cognitive Decline in R6/2 Mice and Increase Longevity

Eighty male and female R6/2 mice and 20 male and female wild-type littermate control mice (F₁ generation) will be bred by Cerebricon at FELASA compliant National Animal Facility Center Kuopio by mating (F₀ generation) WT males (C57BL6CBA F1 hybrid, JAX) with ovarian transferred (01) TG females (JAX). Breeder animals receive igloos instead of play tunnels, nylabone and cotton nestlets and use Purina diet 5008. Upon weaning, pups receive Purina diet 5001.

Female B6CBA F1 hybrid mice (JAX) transplanted with ovaries from R6/2 females are bred with CBA×C57BL/6 F1 WT males (JAX) to generate the transgenic (TG) heterozygous and WT experimental mice. Plugged or visibly pregnant females are removed from the breeding cages to separate housing.

The number of pups nursing per mother should not exceed ten as that is the number of mammary gland nipples available for nursing. When a mother has a litter size of >10 the additional pups should either be cross fostered to another mother with less nursing pups or euthanized. Optimally each mother should be nursing between three and ten pups. Pups in litter sizes of <2 for a mother are generally either euthanized or cross-fostered to mothers with litter sizes <10. Mothers with very small litters (<2) tend to not care well for their pups.

Pups are weaned from their mothers and segregated to new cages for male and females, not exceeding four to five mice per cage. Tail/ear snips are taken during the weaning process at two to three weeks for genotype as described below. Genotyping: Mice are ear marked at the age of 15-21 days and ear/tail samples are collected at the same time for genotyping with PCR. Genotypes are determined between 15 and 21 days (weaning age) of age by PCR of tail snips. In mutant mice, the genotype is a simple PCR assay (see Mangiarini et al. 1997). In general, a 1 to 2 mm snip of tail is biopsied from each animal to be genotyped and snap frozen on dry ice.

### Experimental Set Up of Mice:

For systematic compound testing the following best practices are applied:

In setting up groups for study (i.e., vehicle or drug treated), transgenic and wild-type mice are randomized into groups so that whole litters of mice do not end up in a single testing group. This will avoid “litter effects” on the overall results. In addition, mice are weighed early after weaning and each testing group counterbalanced using mouse body weight.

Mice are housed in groups of four or five and separated by sexes. In each cage, one wild-type mouse of the same gender, but different litter, should be included in an attempt to provide normal social stimulation.

Mice are allowed to acclimate to the experimental room for at least one hour prior to the beginning of any experiment. Mice are transported from the colony room to experimental rooms in their home cages.

Experimentation is conducted in a blinded manner. For instance, the individual dosing mice with vehicle or drug is different from the individual actually running the phenotypic tests. Alternatively, if the same person will do dosing and phenotypic testing, that individual does not have the code for mice receiving drug or vehicle and the vials with vehicle or drug are labeled so as not to allow distinction.

Tail samples are taken at the end of the study for possible verification of genotypes and CAC sizes of individual mice.

Drug Compound Dual-1 (5 or 10 mg/kg), Sertraline (10 mg/kg) or Vehicle is administered i.p. (10 ml/kg) once-a-day (7 to 9 AM) starting at age week 4 and continuing until endpoint. Drug Dual-1 will be delivered by the sponsor as a dry compound and with instructions how to dissolve and prepare the injection solutions. Material safety data sheet or similar document of the compound will be provided by the sponsor if applicable. The solutions are made and stored according to instructions provided by the sponsor (storage conditions and expiration day of solution).

Drug Compound Dual-1 is made fresh daily by diluting to 2.5% DMSO in 20% (2-Hydroxypropyl)-β-cyclodextrin. Sertraline will be made fresh daily by diluting to 1% TWEEN® 80 in ddH2°.

Body weight is measured starting at age of four weeks and two times per week on the same day (i.e., Monday and Thursday) until end of the study. For convenience this is done just prior to animals receiving doses for those days. Animals are monitored twice-a-day by laboratory personnel (8 am and 4 pm) for “survival.” It is most optimal to measure “true survival” that is when the mouse has no detectable heartbeat. Since IACUC restrictions prevent this then using a definable and quantifiable endpoint for “survival” need to be used (see section 2.7 humane end-points). For R6/2 mice such measures can be inconsistent. For instance, body temperature changes can drop just before death but many R6/2 mice show spontaneous death without preceding body temperature changes. More easily measured is body weight loss. For instance defining “survival” endpoint as a 25% or more loss in body weight can be used. Again mice that die spontaneously may not show such acute body weight changes. For these alternative measures combining these “survival” data (body weight or temperature decrease) with “survival” of spontaneous death mice (age found dead in a cage) is suitable.

Two-Choice Swim Test: The two-choice swim test is performed at age week 9. The swim tank (dimensions: 70 cm×30 cm×30 cm) is filled with water (26±1° C.) no higher than 1 cm from the top of a hidden escape platform. The hidden cylindrical platform (diameter, 6.0 cm, height,˜9 cm) is placed either at right or left end of the tank. During acquisition of the task, each mouse is given six training trials per day to swim towards or away from the right end (counterbalanced across subjects) to escape onto the platform. On each trial, mice are released in the center of the tank facing the experimenter and allowed to swim for up to 60 seconds or until they find the platform. A choice is recorded when a mouse moves towards one end and swims beyond 20 cm from the center. If the mouse fails to make a choice within about 15 minutes a “no choice” is recorded. Similarly, if the mouse chooses the goal arm but does not find the platform before leaving the goal arm a “no choice” is recorded. At the end of the trial, once the platform is located or after 60 seconds has elapsed, the mouse is left on the platform for 10 seconds before being returned to its holding cage for an inter-trial interval of about 15 minutes. All animals are trained for four consecutive days. In all trials, choice, latency and “no choice” are recorded.

Compound Dual-1 (5 mg/kg ip) significantly improved the performance of the transgenic mice in the two-choice swim test, as indicated mainly by decreased latency to find the platform at the end of the trial. Also in the swim test, with this lower dose, there was also a clear trend (NS) in the other parameters. See FIG. 6.

### Example 6

### LSD1 Inhibitors and Dual Inhibitors Improve Survival in R6/2 Mice

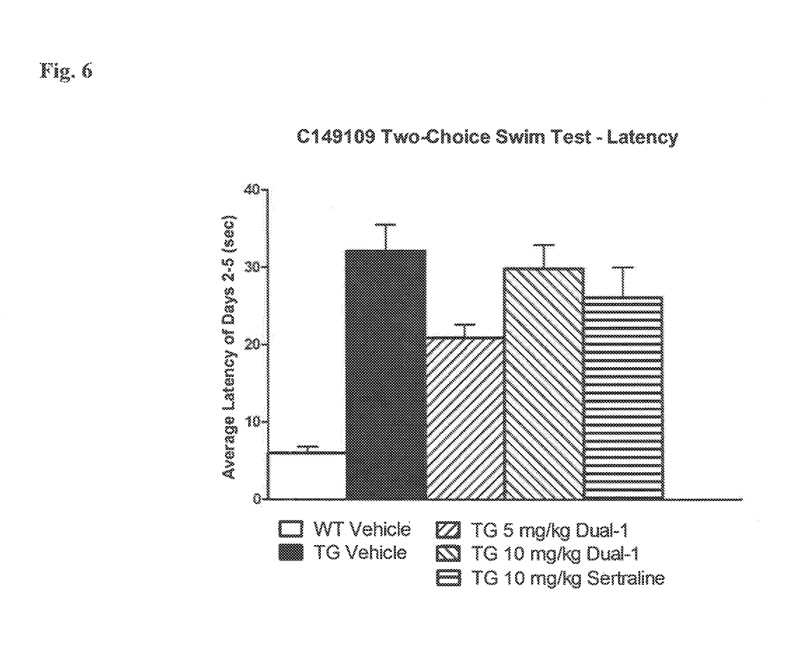

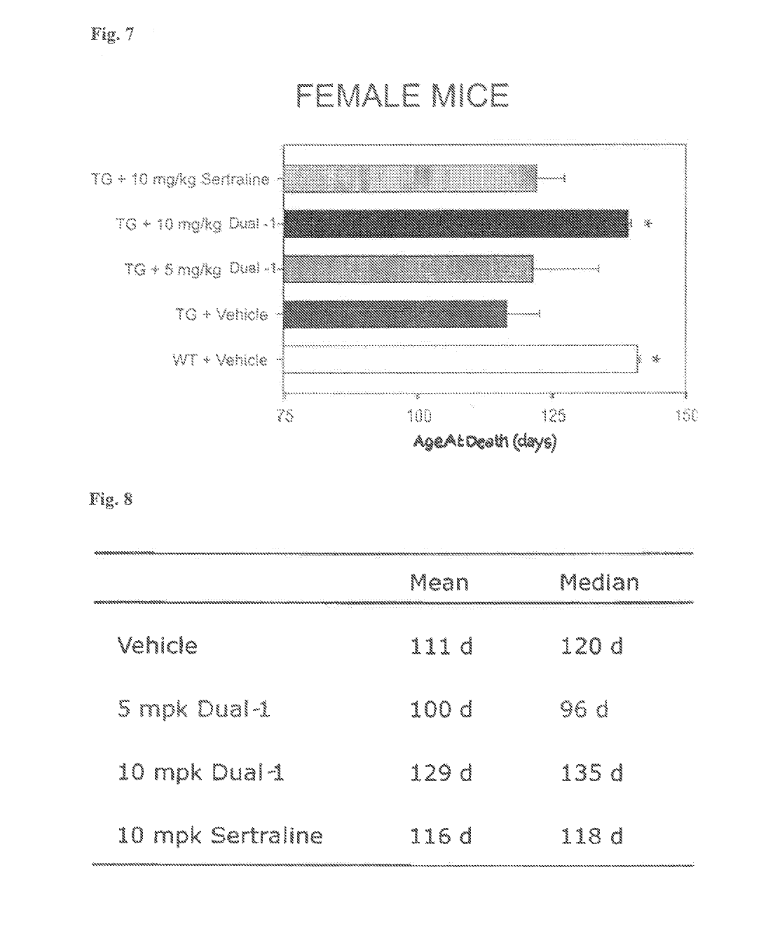

In the R6/2 mouse study described above, the overall survival of the animals were monitored and it was found that dual LSD1/MAO-B inhibitors Compound Dual-1 can increase the longevity in animal expression a gene expected associated with protein conformation disorders. See FIG. 7 and FIG. 8.

### Example 7

### Weight Loss in Chronically/Acutely Treated Animals

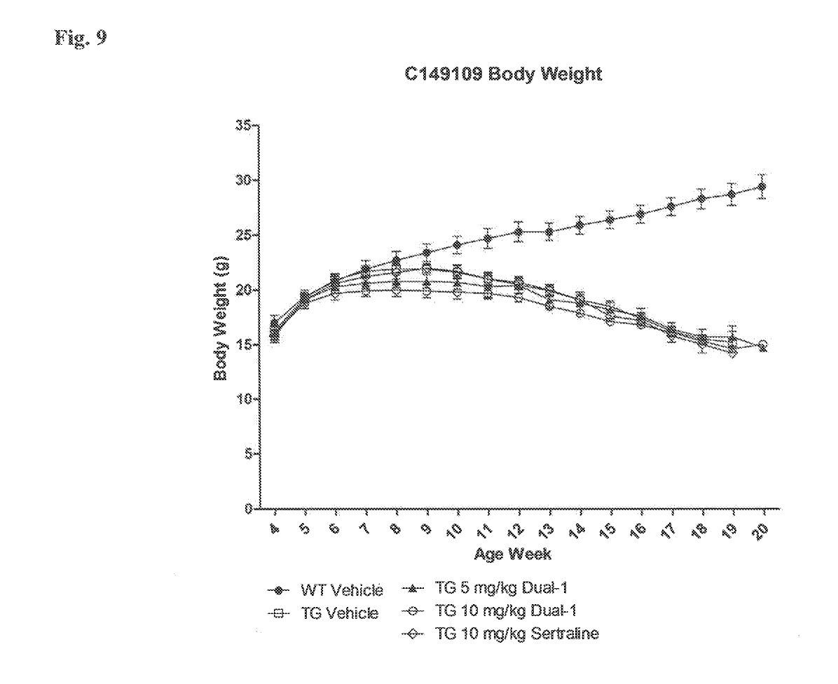

In the R6/2 mouse study described above the weight of the animals were monitored to determine if the LSD1 or LSD1/MAO-B dual inhibitor cause weight loss in chronically treated animals. In particular as seen in FIG. 9 treatment with Compound Dual-1 at either 5 mg/kg or 10 mg/kg IP per day caused no significant weight loss compared to untreated transgenic animal or transgenic animal treated with sertraline indicating that LSD1 selective inhibitors and LSD1/MAO-B inhibitors can be administered safely over periods of times normally used for chronic treatments—from weeks to months of continuous treatment. See FIG. 9.

### Example 8

### Dual LSD1/MAO-B Inhibitors are Effective in Mouse Haloperidol Model of Catalepsy

Dual LSD1/MAO-B inhibitors like Compound Dual-1 were tested in the mouse haloperidol model and found to rescue the toxin induced deficit in a manner similar to or better than control compound caffeine or no treatment. See, e.g., East et al. (2010), Bioorg. Med Chem. Lett., August 15; 20(16):4901-5, Epub 2010 Jun. 25. These experiments indicated that the MAO-B component of the dual inhibitors are effective for treating or preventing motor symptoms of disease.

### Example 9

### Pharmacodynamics, Pharmacokinetics, and Toxicity

Dual LSD1/MAO-B inhibitors and selective LSD1 inhibitors were tested in escalating dose maximum tolerated dose experiments and PK experiments to determine if these targets could be inhibited in vivo in a mammal-like mouse or a human without causing gross toxicity. It was found that dual LSD1/MAO-B inhibitors and selective LSD1 could be dosed in such a manner as to achieve Cmax values above the values expected to achieve pharmacological inhibition of these targets and this was possible to achieve without inducing gross toxicity in mouse. Standard MTD and PK studies available to the skilled artisan were performed to generate these results.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

1-21. (canceled)

22. A method of treating or preventing a cognitive symptom in an individual having a protein conformation disorder comprising identifying an individual in need of such treatment and administering to said individual for a sufficient period of time an amount of an LSD1 inhibitor sufficient to improve the cognitive symptom or reduce the rate of decline of the cognitive symptom thereby treating or preventing said cognitive symptom.

23. The method of claim 22, wherein said protein conformation disorder is a CAG expansion disorder, Alzheimer Disease, or Parkinson Disease.

24-27. (canceled)

28. The method of claim 23, wherein said CAG expansion disorder is Huntington disease, Kennedy Disease, Soinocerebellar Ataxia 1, Spinocerebellar Ataxia 2, Spinocerebellar Ataxia 3, Spinocerebellar Ataxia 6, Soinocerebellar Ataxia 7, or Spinocerebellar Ataxia 17.

29. A method of treating or preventing a motor symptom in an individual having a protein conformation disorder comprising identifying an individual in need of such treatment and administering to said individual for a sufficient period of time an amount of an LSD1 inhibitor sufficient to reduce the rate of decline in said motor symptom thereby treating or preventing said motor symptom.

30. The method of claim 29, wherein said protein conformation disorder is a CAG expansion disorder, Alzheimer Disease, or Parkinson Disease.

31-35. (canceled)

36. A method of increasing longevity in an individual having a protein conformation disorder comprising identifying an individual in need of such treatment and administering to said individual for a sufficient period of time an amount of an LSD1 inhibitor sufficient to increase longevity.

37. The method of claim 36, wherein said protein conformation disorder is a CAG expansion disorder, Alzheimer Disease, or Parkinson Disease.

38-42. (canceled)

43. The method of claim 22, wherein said sufficient period of time is from thirty days to two years.

44. The method of claim 22, wherein said LSD1 inhibitor is administered daily in an amount sufficient to yield a Cmax above the IC50 value for the LSD1 inhibitor.

45. The method of claim 22, wherein said LSD1 inhibitor is administered in an amount from about 0.5 mg to about 500 mg per day.

46. The method of claim 22, wherein said LSD1 inhibitor is an LSD1 selective inhibitor.

47. The method of claim 22, wherein said LSD1 inhibitor is a dual LSD1/MAO-B inhibitor.

48-73. (canceled)

74. The method of claim 22, wherein said LSD1 inhibitor is a 2-cyclylcyclopropan-1-amine compound.

75. The method of claim 22, wherein said LSD1 inhibitor is 2-arylcyclopropan-1-amine compound or a 2-heteroarylcyclopropan-1-amine compound.

76. (canceled)

77. The method of claim 22, wherein said LSD1 inhibitor is a 2-phenylcyclopropan-1-amine compound.

78. The method of claim 22, wherein said LSD1 inhibitor is a 2-cyclylcyclopropan-1-amine compound which is a compound of formula (I) or an enantiomer, a diastereomer or a racemic mixture thereof, or a pharmaceutically acceptable salt or solvate thereof: 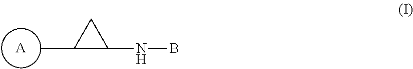
wherein:
A is aryl or heteroaryl optionally having 1, 2, 3, or 4 substituents A′; each A′ is independently selected from -L¹-cyclyl, alkyl, alkenyl, alkynyl, alkoxy, amino, amido, —CH₂—CO—NH₂, alkylamino, hydroxyl, nitro, halo, haloalkyl, haloalkoxy, cyano, sulfonyl, sulfinyl, sulfonamide, acyl, carboxyl, carbamate, and urea, wherein the cyclyl moiety comprised in said -L¹-cyclyl is optionally further substituted with one or more groups independently selected from halo, haloalkyl, haloalkoxy, aryl, arylalkoxy, aryloxy, arylalkyl, alkyl, alkenyl, alkynyl, alkoxy, amino, amido, alkylamino, hydroxyl, nitro, —CH₂—CO—NH₂, heteroaryl, heteroarylalkoxy, heteroaryloxy, heteroarylalkyl, cyano, sulfonyl, sulfinyl, sulfonamide, acyl, carboxyl, carbamate, and urea;
each L¹ is independently selected from a covalent bond, —(CH₂)₁₋₆—, —(CH₂)₀₋₃—O—(CH₂)₀₋₃—, —(CH₂)₀₋₃—NH—(CH₂)₀₋₃—, and —(CH₂)₀₋₃—S—(CH₂)₀₋₃—; B is —H, -L²-CO—NH₂, or -L²-cyclyl, wherein the cyclyl moiety in said -L²-cyclyl is optionally substituted with one or more groups independently selected from halo, haloalkyl, haloalkoxy, haloaryl, aryl, arylalkoxy, aryloxy, arylalkyl, alkyl, alkenyl, alkynyl, alkoxy, amino, amido, alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate, and urea; and
L² is C₁₋₁₂ alkylene which is optionally interrupted by one or more groups independently selected from —O—, —S—, —NH—, —N(alkyl)-, —CO—, —CO—NH—, and —CO—N(alkyl)-, or L² is a covalent bond.

79-80. (canceled)

81. The method of claim 78, wherein A is phenyl optionally having 1, 2, 3, or 4 substituents A′.

82-90. (canceled)

91. The method of claim 78, wherein B is -L²-cyclyl, wherein the cyclyl moiety in said -L²-cyclyl is selected from aryl, cycloalkyl, and heterocyclyl, and further wherein the cyclyl moiety in said -L²-cyclyl is optionally substituted with one or more groups independently selected from halo, haloalkyl, haloalkoxy, haloaryl, aryl, arylalkoxy, aryloxy, arylalkyl, alkyl, alkenyl, alkynyl, alkoxy, amino, amido, alkylamino, hydroxyl, nitro, —CH₂—CO—NH₂, heteroaryl, heteroarylalkoxy, heteroaryloxy, heteroarylalkyl, cycloalkyl, cycloalkylalkoxy, cycloalkoxy, cycloalkylalkyl, heterocycloalkyl, heterocycloalkylalkoxy, heterocycloalkoxy, heterocycloalkylalkyl, cyano, cyanato, isocyanato, thiocyanato, isothiocyanato, sulfonyl, sulfinyl, sulfonamide, trihalomethanesulfonamido, acyl, acylamino, acyloxy, alkylthio, cycloalkylthio, heterocycloalkylthio, arylthio, heteroarylthio, carboxyl, carbamate, and urea.

92-98. (canceled)

99. The method of claim 91, wherein L² is —(CH₂)₁₋₄—, —CH₂—CO—, or a covalent bond.

100-108. (canceled)

109. The method of claim 78, wherein the substituents on the cyclopropane ring are in trans configuration.

110-115. (canceled)
